(19) United States
(12) Patent Application Publication
(10) Pub. No.: US 2003/0072762 A1
(43) Pub. Date: Apr. 17, 2003

(54) Compositions comprising immunostimulatory oligonucleotides and uses thereof to enhance Fc receptor-mediated immunotherapies

(76) Inventors: Jan G. J. van de Winkel, Zeist (NL); George Weiner, Iowa City, IA (US)

Correspondence Address: LAHIVE & COCKFIELD, 28 STATE STREET, BOSTON, MA, 02109, US

(21) Appl. No.: 10/209,070

(22) Filed: Jul. 30, 2002

Related U.S. Application Data
(60) Provisional application No. 60/310,437, filed on Aug. 3, 2001.

(51) Int. Cl. A61K 39/395; A61K 48/00; A61K 39/40

(52) U.S. Cl. 424/178.1; 514/44

(57) ABSTRACT

Compositions comprising immunostimulatory oligonucleotides (CpG ODN) and FcR-directed immunotherapeutics are disclosed. Also disclosed are methods of using the compositions to enhance FcR-mediated antigen presentation, ADCC, and other FcR-mediated immune responses.

### RELATED APPLICATIONS

This application claims priority to U.S. Patent Serial No. 60/310437, filed on Aug. 30, 2001, the contents of which are incorporated herein by reference.

### BACKGROUND OF THE INVENTION

Synthetic oligodeoxynucleotides containing unmethylated CpG motifs (CpG ODN) have been shown to stimulate immune responses.^{5,6,7} For example, CpG ODN activate immune effector cells and induce the production of numerous cytokines.³⁹ In addition, CpG ODN induce growth, activation and maturation of dendritic cells.^{40,41,42} CpG ODN also enhance cytotoxicity against tumor targets. For example, when administered either alone or in combination with a monoclonal antibody, CpG ODN improve immune responses in animal tumor models.^{8,9,10} However, while such therapeutic effects of CpG ODN are known, the mechanisms behind these effects are still poorly understood. Accordingly, a need exists in the art to elucidate these mechanisms and, thereby, to improve CpG ODN-mediated therapies.

The development of monoclonal antibodies (mAbs) has been another valuable addition to current immunotherapies. Recent experience with mAbs, such as Rituximab and Trastuzumab, demonstrate that these drugs are well-tolerated and capable of initiating tumor regression in significant numbers of patients.¹¹⁻¹³ Unfortunately, the vast majority of those treated exhibit only short-lived or partial responses.¹³ Accordingly, there also exists a need to develop technologies for enhancing antibody-mediated immune therapies.

Over the last decade, significant progress has been made in understanding the physiology of Fc receptors (FcRs) and their role in immunity. Fc receptors are crucial for the activity of monoclonal antibodies and are capable of initiating a plethora of biological functions. To date, immunotherapeutic approaches have mainly concentrated on leukocyte FcR for IgG (FcγR).²¹ Three classes of FcγR are currently recognized: FcγRI (CD64); FcγRII (CD32); and FcγRIII (CD16).^{22,23} The human high affinity receptor for IgG, hFcγRI (CD64), is exclusively expressed on cells of the myeloid lineage including monocytes, macrophages, granulocytes (upon cytokine induction) and dendritic cells (DCs).³¹ CD64 is unique among leukocyte FcR because of its cell distribution, structure, and function. CD64 has the capacity to facilitate antigen specific CD4⁺ T cell responses by antigen-presenting cells, and triggers potent anti-tumor vaccine responses.^{32,35} Due to these characteristics this receptor is considered an optimal trigger molecule for antibody therapy.²⁴

Among the types of immune cells expressing CD64, Dendritic cells (DC) are considered promising targets for immunotherapy, as they can trigger naive CD8+T cell responses by their capacity to cross-present exogenous antigens via the major histocompatibility complex (MHC) class I pathway. DCs are professional antigen-presenting cells, with a unique capacity to induce primary immune responses. Tissue residing immature DC exhibit high endocytic and phagocytic activities which, upon maturation, are down-regulated in favor of up regulation of antigen presentation.²⁵ DC-mediated antigen presentation initiates specific immune responses involving both CD4⁺ and CD8⁺ T cell activation. Generally, exogenous antigens are presented on MHC class II molecules and endogenous antigens via the MHC class I pathway. However, cross presentation by DC of exogenous antigens on class I molecules can represent a potent pathway to elicit primary CD8⁺ T cell responses.^{26,3} In principle, cross presentation is an inefficient process as fluid phase internalization of antigens only results in class I-restricted presentation at high concentrations.^{28,5} However, FcγR-mediated uptake of complexed antigens can markedly enhance the efficiency of cross presentation.³⁰

### SUMMARY OF THE INVENTION

The present invention provides compositions and methods for enhancing FcR-mediated immunotherapies. In particular, as part of the present invention, it was discovered that immunostimulatory CpG-containing oligonucleotides (CpG ODN) augment FcR-mediated immune responses, particularly FcγRI (CD64)-mediated immune responses. Accordingly, the compositions and methods of the present invention include FcR-directed compounds in combination with immunostimulatory oligonucleotides.

For example, as demonstrated in the studies described herein, immunostimulatory oligonucleotides can be used to enhance antibody dependent cellular cytotoxicity (ADCC) induced by FcR-targeted antibodies, including bispecific antibodies. Thus, in one embodiment, the invention provides a composition comprising one or more immunostimulatory oligonucleotides in combination with a monoclonal antibody or FcR-targeted bispecific or multispecific antibody directed against a target cell or pathogen. Typical target cells against which ADCC is induced include, but are not limited to, tumor cells such as cells from ovarian, breast, testicular and prostate tumors, as well as leukemia and lymphoma. Typical target pathogens include, for example, viruses and bacteria.

The bispecific and multispecific antibodies used in the invention bind to a target cell or pathogen, and to an Fc receptor (e.g., a human Fc receptor), such that they induce FcR-mediated ADCC of the target cell or pathogen by an effector cell, e.g., a monocyte, macrophage or an activated polymorphonuclear cell. Preferred Fc receptors for targeting include Fc-gamma receptors (FcγRs), particularly FcγRI (CD64), but also FcγRII (CD32), and FcγRIII (CD16). However, other Fc receptors, such as IgA receptors (e.g. FcαRI), also can be targeted. In a particular embodiment, the bispecific or multispecific antibody binds to an Fc receptor at a site which is distinct from the natural ligand binding site of the receptor (i.e., the immunoglobulin Fc (e.g., IgG or IgA) binding site of the receptor). Therefore, the binding of the bispecific or multispecific antibody is not blocked by physiological levels of immunoglobulins.

The bispecific and multispecific antibodies of the invention, in one embodiment, include two or more antibodies or antibody fragments (e.g., an Fab, Fab′, F(ab′)₂, Fv, or a single chain Fv), linked together either chemically or genetically. Preferred antibodies include fully human monoclonal antibodies, as well as “chimeric” and “humanized” antibodies. Murine monoclonal antibodies also can be used.

Accordingly, in another aspect, the invention provides a method of enhancing FcR-mediated ADCC or killing (e.g., lysing or phagocytosis) of a target cell, such as a cancer cell, in a subject by administering to the subject a composition comprising one or more immunostimulatory oligonucleotides and a bispecific antibody directed against the target cell and an Fc receptor, such as CD64. The method can be used to treat a variety of diseases, particularly cancers, by inhibiting or preventing the growth of target (e.g., tumor) cells.

As also demonstrated in the studies described herein, immunostimulatory oligonucleotides can be used to enhance Fc receptor-mediated antigen presentation. For example, it is shown herein that immunostimulatory oligonucleotides can increase dendritic cell (DC)-mediated cross presentation (MHC Class I presentation) of CD64-targeted antigens.

Accordingly, in yet another embodiment, the invention provides a vaccine composition comprising one or more immunostimulatory oligonucleotides in combination with an FcR-targeted antigen. Suitable antigens include any antigen against which an increased immune response is desired (e.g., any antigen which can be used as a vaccine). Typical antigens include tumor, viral and bacterial antigens.

The FcR-targeted antigen is targeted to an FcR by way of linking the antigen to a moiety that binds to an FcR on an antigen presenting cell (APC), such that the antigen is targeted to the cell. The moiety that binds to the FcR is typically an antibody or antibody fragment that binds to FcR. For example, the FcR-targeted antigen can be a fusion protein or molecular conjugate containing the antigen linked (e.g., chemically or genetically) to an antibody or antibody fragment which binds to an FcR. In a particular embodiment, the FcR-targeted antigen comprises a single chain fusion protein comprising the antigen linked to a single chain antibody directed against an FcR, such as CD64.

Accordingly, in another aspect, the invention provides a method of enhancing Fc receptor-mediated (e.g., CD64-mediated) antigen presentation, such as dendritic cell (DC)-mediated cross presentation (MHC Class I presentation) of CD64-targeted antigens, in a subject by administering to the subject one or more immunostimulatory oligonucleotides in combination with an FcR-targeted antigen. By enhancing antigen presentation in this manner, the therapeutic efficacy of CD64-directed tumor vaccines can be augmented and antigen-specific antibody responses can be induced. Thus, a variety of diseases can be treated and/or prevented including, but not limited to, cancers, autoimmune diseases, and pathogenic (e.g., viral and bacterial) infections.

Therapeutic compositions of the present invention can be formulated in a pharmaceutically acceptable carrier and administered to a subject using any suitable route of administration. Typically the compositions are administered by injection, in an appropriate amount and dosage regimen to achieve a therapeutic effect. In all embodiments of the invention, the immunostimulatory oligonucleotide(s) can be formulated together with the FcR-targeted molecule (e.g., antigen or tumor-directed bispecific antibody) in a single composition such that they are co-administered, or alternatively, the immunostimulatory oligonucleotide(s) and the FcR-targeted molecule can be formulated separately as two distinct compositions. In this instance, the separate compositions can be administered together (concurrently) or can be administered separately (sequentially).

Therapeutic compositions of the present invention also can be coadministered with other therapeutic and cytotoxic agents. For example, they can be coadministered with chemotherapeutic agents such as doxorubicin (adriamycin), cisplatin bleomycin sulfate, carmustine, chlorambucil, and cyclophosphamide hydroxyurea. The compositions of the invention also can be administered in conjunction with radiation therapy. The compositions of the invention also can be administered in conjunction with an agent that modulates, e.g., enhances or inhibits, the expression or activity of an Fc receptor, e.g., an Fcα receptor or an Fcγ receptor, such as a cytokine. Typical cytokines for administration during treatment include granulocyte colony-stimulating factor (G-CSF), granulocyte- macrophage colony-stimulating factor (GM-CSF), interferon-γ (IFN-γ), and tumor necrosis factor (TNF). Typical therapeutic agents include, among others, anti-neoplastic agents such as doxorubicin (adriamycin), cisplatin bleomycin sulfate, carmustine, chlorambucil, and cyclophosphamide hydroxyurea.

Other features and advantages of the instant invention be apparent from the following detailed description and claims.

### BRIEF DESCRIPTION OF THE DRAWINGS

FIG. 1 is a graph showing the enhanced effect of CpG ODN on murine IgG2a-induced immunotherapy against a B cell lymphoma. Groups of 6 C3H/HeN mice were inoculated i.p. with 38C13 T3C tumor cells on day 0. Consequently, mice were treated once daily (on days 5, 7, and 10) with 100 μg mAb (IgG1 or IgG2a) alone, 20 μg CpG ODN 1826, or a combination. Treatment schedule is shown on the right. Survival was recorded daily. Similar results were obtained in three independent experiments.

FIGS. 2A and B are graphs showing the effect of CpG ODN on PMN hFcγRl (CD64) expression levels. hFcγRI-Tg and NTg mice were treated with single doses of 5, 7.5, or 10 μg CpG ODN 1826 s.c. and mice treated with saline (0.9% NaCl) served as controls. hFcγRI expression levels on PMN were determined by FACS analysis of whole blood. PMN expression levels of hFcγRI of Tg mice, Tg mice treated with 7.5 pg CpG ODN 1826, and NTg mice are shown in panel A. hFcγRJ expression levels of Tg mice treated with different CpG ODN concentrations are shown in panel B. Control FITC-labeled murine IgG overlapped with the curve of NTg mice in panel A. Experiment was repeated three times, yielding similar results.

FIG. 3 is a graph showing the kinetics of CpG ODN on hFcγRl expression. hFcγRl-Tg and Ntg mice were injected with a single s.c. dose of 7.5 μg CpG ODN 1826, and Tg mice treated with saline served as controls. hFcγRI expression levels were determined on five consecutive days by FACS analysis of whole blood. hFcγRI expression levels at the indicated time points are shown. Level of irrelevant murine IgG was identical to the level of the NTg mice in FIG. 1A. Similar results were obtained in three independent experiments.

FIGS. 4A-F are graphs showing the in vivo effect of CpG ODN on leukocytes. hFcγRI-Tg and NTg mice were treated with 7.5 μg of CpG ODN 1826 and mice treated with saline served as controls. Whole blood was analyzed by FACS using lineage-specific markers (see Material and Methods). Data shown in the different panels represent percentages of cells at five consecutive time-points for granulocytes (A), monocytes ( B), DC (C), T cells (D), and B cells (E). In addition, the expression of murine FcγRII/III was determined (F), Tg+saline (open square), Tg+CpG ODN 1826 (closed square), NTg+saline (open triangle), NTg+CpG ODN 1826 (closed triangle). Similar results were obtained in three independent experiments.

FIG. 5 is a graph showing the effect of CpG ODN on hFcγRl-mediated ADCC. SK-Br-3 cells labeled with ⁵¹Cr were incubated with different concentrations of BsAb MDX-H210 and 10 μg/ml CpG ODN 1826 or 1982. Medium without mAb, anti-HER-2/neu mAb 520C9 (mIgG1; 2 μg/ml), or CpG ODN alone (1826 or 1982; 10 μg/ml) served as controls. Tg and NTg mice were treated with G-CSF for three days prior to the start of the experiment to increase granulocyte numbers. All determinations were performed in triplicate and similar results were obtained in three independent experiments.

FIG. 6 is a graph showing the enhanced effect of CpG ODN on hFcγRI-directed solid tumor immunotherapy. Groups of 6 hFcγRl-Tg and NTg mice received 7.5 μg CpG ODN 1826 s.c. on day −1. On day 0, CMS7HE tumor cells were inoculated s.c. in the right flank. Consequently, they were treated twice daily i.p. (on days 1-5, and 9-13) with 10 μg MDX-H210 or 100 μl PBS. On day 7, the second dose of CpG ODN 1826 was administered. Treatment schedule is shown on the right. Tumor volumes were measured three times a week and animals were scored for toxocity. Similar results were obtained in three independent experiments.

FIGS. 7A-C are graphs showing the effect of culture conditions on DC7 cell surface marker expression. Murine CD64-Tg and NTg DC were cultured for 7 days either in the presence of GM-CSF (A), or with GM-CSF/TNF-α (B). DC7 were cultured for 2 additional days in the presence of LPS (L) to study DC maturation (C). Expression of specific markers was analyzed by flow cytometry. Controls are depicted as open histograms and monoclonal antibodies (mAb) as filled histograms in A and B. In C, controls are depicted as closed histograms, and mAb as gray lines (no LPS) or black lines (with LPS). One representative experiment out-of-three is shown.

FIG. 8 is a graph showing the MHC class II antigen-presenting capacity of DC. Human CD64-Tg DC, cultured for 7 or 9 days in the presence of either GM-CSF or GM-CSF/TNF-α, were incubated with excess OVA (400 μg/ml), or with OVA-IgGαOVA complexes (100 ng/ml), and OVA-specific MHC class II-restricted DO11.10 T cells for 24 hat 37° C. Levels of IL-2 production by T cells were determined by CTLL-2 proliferation assays. Data represent means of duplicate determinations in one representative experiment out-of-four.

FIG. 9 shows the effect of CpG ODN on DC cell surface marker expression. Human CD64-Tg DC were cultured for 7 days in the presence of GM-CSF or GM-CSF/TNF-α. Subsequently, CpG ODN (100 μg/ml) were added for 24 h. Cell surface expression of different markers was analyzed by flow cytometry. Data shown are representative of three independent experiments, yielding identical results.

FIG. 10 shows the effect of CpG ODN on MHC class I presentation. Human CD64-Tg DC were cultured for 7 days in the presence of either GM-CSF or GM-CSF/TNF-α. These DC were incubated with either 124 μg/ml SIINFEKL (A), or with different concentrations of OVA-IgGαOVA immune complexes (B), in the presence or absence of CpG ODN for 24 h at 37° C. Cells were fixed, washed and incubated with MHC class I-restricted OVA-specific RF33 T cells for 24 h at 37° C. IL-2 released by T cells was determined by CTLL-2 proliferation assays. One representative experiment out-of-four is shown.

FIG. 11 shows the effect of CpG ODN on human CD64-mediated cross presentation. Human CD64-Tg and NTg DC were cultured for 7 days in the presence of either GM-CSF (A, B), or GM-CSF/TNF-α (C, D), and were then incubated with 22-OVA (genetically engineered) (A, C) or 22×OVA (chemically cross-linked) (B, D), either with or without CpG ODN, for 24 h at 37° C. Cells were fixed, washed and incubated with MHC class I-restricted OVA-specific RF33 T cells for 24 h at 37° C. IL-2 released by T cells was determined by CTLL-2 proliferation assays. One representative experiments out-of-three is shown.

### DETAILED DESCRIPTION OF THE INVENTION

The present invention uses immunostimulatory CpG-containing oligonucleotides (CpG ODN) to augment FcR-mediated immune responses, particularly FcγRI (CD64)-mediated immune responses. By coadministering the immunostimulatory CpG-containing oligonucleotide with FcR-targeted immunotherapeutic agents, the therapeutic effect of the agent is enhanced. This is based on the discovery, as part of the present invention, that immunostimulatory CpG-containing oligonucleotides increase CD64 expression and stimulate effector cells, including phagocyte proliferation.

In order that the present invention may be more readily understood, the following terms are defined as follows:

The terms “immunostimulatory oligonucleotide”, “immunostimulatory CpG containing oligonucleotide” and “CpG ODN” are used interchangeably herein, and all refer to an oligonucleotide which contains a cytosine, guanine nucleotide sequence, which is capable of increasing an FcR-mediated immune response including but not limited to FcR-mediated ADCC, particularly FcγRI-mediated ADCC, and FcR-mediated antigen presentation, particularly FcγRI-mediated antigen presentation. Preferred immunostimulatory oligonucleotides are between 2 and 100 base pairs in size, more preferably between 10 and 50 base pairs, and most preferably between 15-25 base pairs (e.g., about 20 base pairs) in length.

Immunostimulatory oligonucleotides for use in the present invention can be prepared as described in U.S. Pat. No. 6,194,388, the entire contents of which is hereby incorporated by reference herein. As described therein, immunostimulatory oligonucleotides contain a consensus mitogenic CpG motif represented by the formula: ^{5′}X₁X₂CGX₃X₄^{3′} wherein C and G are unmethylated, X₁, X₂, X₃, and X₄ are nucleotides and a GCG trinucleotide sequence is not present at or near the 5′ and 3′ termini.

The term “bispecific molecule” is intended to include any agent, e.g., a protein, peptide, or protein or peptide complex, which has two different binding specificities. For example, the molecule may bind to, or interact with, (a) a cell surface antigen, such as a tumor antigen, and (b) an Fc receptor on the surface of an effector cell, e.g., FcRγRI (CD64). Typical bispecific molecules include “bispecific antibodies” which are composed of two antibodies or antibody fragments having different binding specificities linked together. The term “multispecific molecule” or “heterospecific molecule” is intended to include any agent, e.g., a protein, peptide, or protein or peptide complex, which has two or more different binding specificities. Accordingly, “multispecific molecules” include bispecific molecules, as well as molecules which have more than two binding specificities. For example, the molecule may bind to, or interact with, (a) a cell surface antigen, (b) an Fc receptor on the surface of an effector cell, and (c) at least one other component. Accordingly, the invention includes, but is not limited to, bispecific, trispecific, tetraspecific, and other multispecific molecules which are directed to cell surface antigens and to other targets, such as Fc receptors on effector cells.

“Bispecific antibodies” also include diabodies. Diabodies are bivalent, bispecific antibodies in which the VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123).

As previously described herein, multispecific molecules (e.g., bispecific antibodies) for use in the present invention are directed against Fc receptors (e.g., have one or more binding specificities for an Fc receptor), preferably Fc gamma receptors, such as CD64. Such Fc gamma-directed bispecific molecules and bispecific antibodies can be generated as described in U.S. Pat. No. 5,635,600, the entire contents of which is hereby incorporated by reference herein. Bispecific molecules directed against Fc alpha receptor (CD89) for use in the present invention can be prepared as described in U.S. Pat. No. 6,193,966, the entire contents of which is hereby incorporated by reference herein. Other bispecific molecules which can be used in the present invention are described in U.S. Pat. No. 5,837,243, the entire contents of which is hereby incorporated by reference herein.

In one embodiment, the binding specificity for an Fc receptor is provided by a human monoclonal antibody against FcγRI, the binding of which is not blocked by human immunoglobulin G (IgG). The production and characterization of these preferred monoclonal antibodies are described by Fanger et al. in PCT application WO 88/00052 and in U.S. Pat. No. 4,954,617, the teachings of which are fully incorporated by reference herein. These antibodies bind to an epitope of FcγRI, FcγRII or FcγRIII at a site which is distinct from the Fcγ binding site of the receptor and, thus, their binding is not blocked substantially by physiological levels of IgG. Specific anti-FcγRI antibodies useful in this invention are mAb 22, mAb 32, mAb 44, mAb 62 and mAb 197. The hybridoma producing mAb 32 is available from the American Type Culture Collection, ATCC Accession No. HB9469. Anti-FcγRI mAb 22, F(ab′)₂ fragments of mAb 22, and can be obtained from Medarex, Inc. (Annandale, N.J.). In other embodiments, the anti-Fcγ receptor antibody is a humanized form of monoclonal antibody 22 (H22). The production and characterization of the H22 antibody is described in Graziano, R. F. et al. (1995) J. Immunol 155 (10): 4996-5002 and PCT/US93/10384. The H22 antibody producing cell line was deposited at the American Type Culture Collection on Nov. 4, 1992 under the designation HA022CL1 and has the accession no. CRL 11177.

In still other preferred embodiments, the binding specificity for an Fc receptor is provided by an antibody that binds to a human IgA receptor, e.g., an Fc-alpha receptor (FcαRI (CD89)), the binding of which is preferably not blocked by human immunoglobulin A (IgA). Four FcαRI-specific monoclonal antibodies, identified as A3, A59, A62 and A77, which bind FcαRI outside the IgA ligand binding domain, have been described (Monteiro, R. C. et al., 1992, J. Immunol. 148:1764).

FcαRI and FcγRI are preferred trigger receptors for use in the present invention because they are (1) expressed primarily on immune effector cells, e.g., monocytes, PMNs, macrophages and dendritic cells; (2) expressed at high levels (e.g., 5,000-100,000 per cell); (3) mediators of cytotoxic activities (e.g., ADCC, phagocytosis); (4) mediate enhanced antigen presentation of antigens, including self-antigens, targeted to them.

The term “antibody”, as used herein, includes whole antibodies and any antigen binding fragment (i.e., “antigen-binding portion”) or single chain thereof. An “antigen-binding fragment” of an antibody, as used herein, refers to one or more fragments of an antibody that retains the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

The terms “monoclonal antibody” and “monoclonal antibody composition” as used herein refer to a preparation of antibody molecules of single molecular composition. A monoclonal antibody composition displays a single binding specificity and affinity for a particular epitope. Accordingly, the term “human monoclonal antibody” refers to antibodies displaying a single binding specificity which have variable and constant regions derived from human germline immunoglobulin sequences. In one embodiment, the human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell.

Human antibodies can be generated as described in U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay, and GenPharm International; U.S. Pat. No. 5,545,807 to Surani et al.; International Publication Nos. WO 98/24884, published on Jun. 11, 1998; WO 94/25585, published Nov. 10, 1994; WO 93/1227, published Jun. 24, 1993; WO 92/22645, published Dec. 23, 1992; WO 92/03918, published Mar. 19, 1992, the disclosures of all of which are hereby incorporated by reference in their entity.

The terms “enhance”, “augment” and “increase” as referring to FcR-mediated immune responses and FcR-mediated antigen presentation are used interchangeably herein, and include any level of increase in an immune response (e.g., ADCC, cellular lysis, phagocytosis, antibody production and opsonization, cytokine production etc.) or in antigen presentation when an FcR-directed therapeutic of the present invention is administered in conjunction with immunostimulatory oligonucleotides as compared to when it is administered in the absence of immunostimulatory oligonucleotides.

As used herein, the term “inhibits growth” (e.g., referring to cells) is intended to include any measurable decrease in the growth of a cell as compared to a control cell, e.g., the inhibition of growth of a cell by at least about 10%, 20%, 30%, 40%, 50%, 60%, 70%, 80%, 90%, 99%, or 100%.

As used herein, the terms “inhibits binding” and “blocks binding” (e.g., referring to inhibition/blocking of a cellular ligand to its receptor) are used interchangeably and encompass both partial and complete inhibition/blocking, e.g., by at least about 10%, 20%, 30%, 40%, 50%, 60%, 70%, 80%, 90%, 99%, or 100%.

The term “nucleic acid molecule”, as used herein, is intended to include DNA molecules and RNA molecules. A nucleic acid molecule may be single-stranded or double-stranded, but preferably is double-stranded DNA. Thus, immunostimulatory oligonucleotides are short nucleic acid molecules.

For nucleic acids, the term “substantial homology” indicates that two nucleic acids, or designated sequences thereof, when optimally aligned and compared, are identical, with appropriate nucleotide insertions or deletions, in at least about 80% of the nucleotides, usually at least about 90% to 95%, and more preferably at least about 98% to 99.5% of the nucleotides. Alternatively, substantial homology exists when the segments will hybridize under selective hybridization conditions, to the complement of the strand.

The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

As used herein, the term “effector cell” refers to an immune cell which is involved in the effector phase of an immune response, as opposed to the cognitive and activation phases of an immune response. Exemplary immune cells include a cell of a myeloid or lymphoid origin, e.g., lymphocytes (e.g., B cells and T cells including cytolytic T cells (CTLs)), killer cells, natural killer cells, macrophages, monocytes, eosinophils, neutrophils, polymorphonuclear cells, granulocytes, mast cells, and basophils. Some effector cells express specific Fc receptors and carry out specific immune functions. In preferred embodiments, an effector cell is capable of inducing antibody-dependent cell-mediated cytotoxicity (ADCC), e.g., a neutrophil capable of inducing ADCC. For example, monocytes, macrophages, which express FcR are involved-in specific killing of target cells and-presenting antigens to other components of the immune system, or binding to cells that present antigens. In other embodiments, an effector cell can phagocytose a target antigen, target cell, or microorganism. The expression of a particular FcR on an effector cell can be regulated by humoral factors such as cytokines. For example, expression of FcγRI has been found to be up-regulated by interferon gamma (IFN-γ). This enhanced expression increases the cytotoxic activity of FcγRI-bearing cells against targets. An effector cell can phagocytose or lyse a target antigen or a target cell.

As used herein, the term “target cell” refers to any undesirable cell in a subject (e.g., a human or animal) that can be targeted by a composition (e.g., a human monoclonal antibody, a bispecific or a multispecific molecule) of the invention. In particular embodiments, the target cell is a cell expressing or overexpressing a tumor antigen, such as bladder, breast, colon, kidney, ovarian, prostate, renal cell, squamous cell, lung (non-small cell), and head and neck tumor cells. Other target cells include synovial fibroblast cells.

While human monoclonal antibodies are preferred, other antibodies which can be employed in the bispecific or multispecific molecules of the invention are murine, chimeric and humanized monoclonal antibodies.

Chimeric mouse-human monoclonal antibodies (i.e., chimeric antibodies) can be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted. (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

The chimeric antibody can be further humanized by replacing sequences of the Fv variable region which are not directly involved in antigen binding with equivalent sequences from human Fv variable regions. General reviews of humanized chimeric antibodies are provided by Morrison, S. L., 1985, Science 229:1202-1207 and by Oi et al., 1986, BioTechniques 4:214. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from 7E3, an anti-GPII_{b}IIIₐ antibody producing hybridoma. The recombinant DNA encoding the chimeric antibody, or fragment thereof, can then be cloned into an appropriate expression vector. Suitable humanized antibodies can alternatively be produced by CDR substitution U.S. Pat. No. 5,225,539; Jones et al. 1986 Nature 321:552-525; Verhoeyan et al. 1988 Science 239:1534; and Beidler et al. 1988 J. Immunol. 141:4053-4060.

All of the CDRs of a particular human antibody may be replaced with at least a portion of a non-human CDR or only some of the CDRs may be replaced with non-human CDRs. It is only necessary to replace the number of CDRs required for binding of the humanized antibody to the Fc receptor.

An antibody can be humanized by any method, which is capable of replacing at least a portion of a CDR of a human antibody with a CDR derived from a non-human antibody. Winter describes a method which may be used to prepare the humanized antibodies of the present invention (UK Patent Application GB 2188638A, filed on Mar. 26, 1987), the contents of which is expressly incorporated by reference. The human CDRs may be replaced with non-human CDRs using oligonucleotide site-directed mutagenesis as described in International Application WO 94/10332 entitled, Humanized Antibodies to Fc Receptors for Immunoglobulin G on Human Mononuclear Phagocytes.

Also within the scope of the invention are chimeric and humanized antibodies in which specific amino acids have been substituted, deleted or added. In particular, preferred humanized antibodies have amino acid substitutions in the framework region, such as to improve binding to the antigen. For example, in a humanized antibody having mouse CDRs, amino acids located in the human framework region can be replaced with the amino acids located at the corresponding positions in the mouse antibody. Such substitutions are known to improve binding of humanized antibodies to the antigen in-some instances. Antibodies in which amino acids have been added, deleted, or substituted are referred to herein as modified antibodies or altered antibodies.

The term modified antibody is also intended to include antibodies, such as monoclonal antibodies, chimeric antibodies, and humanized antibodies which have been modified by, e.g., deleting, adding, or substituting portions of the antibody. For example, an antibody can be modified by deleting the constant region and replacing it with a constant region meant to increase half-life, e.g., serum half-life, stability or affinity of the antibody. Any modification is within the scope of the invention so long as the bispecific and multispecific molecule has at least one antigen binding region specific for an FcγR and triggers at least one effector function.

Bispecific and multispecific molecules of the present invention can be made using chemical techniques (see e.g., D. M. Kranz et al. (1981) Proc. Natl. Acad. Sci. USA 78:5807), “polydoma” techniques (See U.S. Pat. No. 4,474,893, to Reading), or recombinant DNA techniques.

In particular, bispecific and multispecific molecules of the present invention can be prepared by conjugating the constituent binding specificities, e.g., the anti-FcR and anti-HER-2/neu binding specificities, using methods known in the art and described in the examples provided herein. For example, each binding specificity of the bispecific and multispecific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl) cyclohaxane-1-carboxylate (sulfo-SMCC) (see e.g., Karpovsky et al. (1984) J. Exp. Med. 160:1686; Liu, M A et al. (1985) Proc. Natl. Acad. Sci. USA 82:8648). Other methods include those described by Paulus (Behring Ins. Mitt. (1985) No. 78, 118-132); Brennan et al. (Science (1985) 229:81-83), and Glennie et al. (J. Immunol. (1987) 139: 2367-2375). Preferred conjugating agents are SATA and sulfo-SMCC, both available from Pierce Chemical Co. (Rockford, Ill.).

When the binding specificities are antibodies (e.g., two humanized antibodies), they can be conjugated via sulfhydryl bonding of the C-terminus hinge regions of the two heavy chains. In a particularly preferred embodiment, the hinge region is modified to contain an odd number of sulfhydryl residues, preferably one, prior to conjugation.

Alternatively, both binding specificities can be encoded in the same vector and expressed and assembled in the same host cell. This method is particularly useful where the bispecific and multispecific molecule is a mAb×mAb, mAb×Fab, Fab×F(ab′)₂ or ligand×Fab fusion protein. A bispecific and multispecific molecule of the invention, e.g., a bispecific molecule can be a single chain molecule, such as a single chain bispecific antibody, a single chain bispecific molecule comprising one single chain antibody and a binding determinant, or a single chain bispecific molecule comprising two binding determinants. Bispecific and multispecific molecules can also be single chain molecules or may comprise at least two single chain molecules. Methods for preparing bi- and multspecific molecules are described for example in U.S. Pat. No. 5,260,203; U.S. Pat. No. 5,455,030; U.S. Pat. No. 4,881,175; U.S. Pat. No. 5,132,405; U.S. Pat. No. 5,091,513; U.S. Pat. No. 5,476,786; U.S. Pat. No. 5,013,653; U.S. Pat. No. 5,258,498; and U.S. Pat. No. 5,482,858.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., antibody, bispecific and multispecific molecule, may be coated in a material to protect the compound from the action of acids and other natural conditions that may inactivate the compound.

Compositions of the present invention can be administered by a variety of methods known in the art. As will be appreciated by the skilled artisan, the route and/or mode of administration will vary depending upon the desired results. The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

To administer compounds of the invention by certain routes of administration, it may be necessary to coat the compound with, or co-administer the compound with, a material to prevent its inactivation. For example, the compound may be administered to a subject in an appropriate carrier, for example, liposomes, virosomes, or a diluent. Pharmaceutically acceptable diluents include saline and aqueous buffer solutions. Liposomes include water-in-oil-in-water CGF emulsions as well as conventional liposomes (Strejan et al. (1984) J. Neuroimmunol. 7:27).

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

For the therapeutic compositions, formulations of the present invention include those suitable for oral, nasal, topical (including buccal and sublingual), rectal, vaginal and/or parenteral administration. The formulations may conveniently be presented in unit dosage form and may be prepared by any methods known in the art of pharmacy. The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred per cent, this amount will range from about 0.01 per cent to about ninety-nine percent of active ingredient, preferably from about 0.1 per cent to about 70 per cent, most preferably from about 1 per cent to about 30 per cent.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

When the compounds of the present invention are administered as pharmaceuticals, to humans and animals, they can be given alone or as a pharmaceutical composition containing, for example, 0.01 to 99.5% (more preferably, 0.1 to 90%) of active ingredient in combination with a pharmaceutically acceptable carrier.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved. In general, a suitable daily dose of a compositions of the invention will be that amount of the compound which is the lowest dose effective to produce a therapeutic effect. Such an effective dose will generally depend upon the factors described above. It is preferred that administration be intravenous, intramuscular, intraperitoneal, or subcutaneous, preferably administered proximal to the site of the target. If desired, the effective daily dose of a therapeutic compositions may be administered as two, three, four, five, six or more sub-doses administered separately at appropriate intervals throughout the day, optionally, in unit dosage forms. While it is possible for a compound of the present invention to be administered alone, it is preferable to administer the compound as a pharmaceutical formulation (composition).

Therapeutic compositions can be administered with medical devices known in the art. For example, in a preferred embodiment, a therapeutic composition of the invention can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

The present invention is further illustrated by the following examples which should not be construed as further limiting. The contents of all figures and all references, patents and published patent applications cited throughout this application are expressly incorporated herein by reference. Those skilled in the art will recognize, or be able to ascertain using no more than routine experimentation, many equivalents to the specific embodiments of the invention described herein. Such equivalents are intended to be encompassed by the following Examples and claims.

### EXAMPLES

Part I—Immunostimulatory Oligonucleotides Increase FcR (CD64) Expression and Enhance CD64-Mediated Tumor Cell Killing

Materials and Methods

Mice: FVB/N mice transgenic for hFcγRI were crossed back into Balb/C.³⁰ F12 littermates and C3H/HeN mice were used for these studies. In all experiments with hFcγRI-Tg animals, the Tg mice were matched with their non-transgcnic (NTg) littermates. Female CH3/HeN mice were obtained from Harlan-Sprague-Dawley (Indianapolis, Ind.). Mice were bred and maintained either in the Transgenic Mouse Facility of the Centtal Laboratory Animal Facility (Utrecht, The Netherlands) or in the Animal Care Unit at the University of Iowa (Iowa City, Iowa) and were used at 8-16 weeks of age. All experiments were approved by the Utrecht, or University of Iowa animal ethics committees.

Cell lines: SK-BR-3, a breast carcinoma cell line over-expressing HER-2/neu, was obtained from the American Type Culture Collection (A TCC, Manassas, Va., HTB-30).³¹ Cells were cultured in medium consisting of RPMI 1640 medium (Gibco BRL, Life Technologies, Paisley, Scotland), supplemented with 10% heat-inactivated fetal bovine serum (FBS) (Hyclone, Logan, Utah), 50 μg/ml streptomycin, 50 IU/ml penicillin. and 4 mM L-glutamine (all Gibco BRL), hereby called complete medium. The 3-methylcholantrene-induced murine fibrosarcoma cell line, CMS7HE, stably transfected with human HER-2/neu, together with a control cell line, transfected with an empty vector, CMS7neo, were provided by Dr. Hiroshi Shiku (Mie University School of Medicine, Mie, Japan).^{32,33} These cells were maintained in complete medium, supplemented with 462 μg/ml Geneticin (G418 sulphate; Gibco BRL). The murine B cell lymphoma cell line 38C13 DC, a sub-clone of the original 38C13 cell line, was maintained in complete medium supplemented with 2-mercaptoethanol. This cell line has been extensively used in anti-tumor animal models.^{34,35} All adherent cell lines were detached by using trypsin-EDTA (Life Technologies) in phosphate-buffered saline (PBS). Cells used for tumor inoculation were collected in log-phase and were tested for stable HER-2/neu expression by FACS analysis and for potential mycoplasm contamination, before each experiment.

Antibodies: A panel of anti-mouse mAb, unlabeled or labeled with either fluorescein isothiocyanate (FITC)-, R-phycoerythrin (RPE), or biotin, was used to detect the different murine effector cell populations. CD3-FITC (clone 17-2a; rat IgG2bκ), CD4-RPE (rat IgG2bκ), CD8a-FITC (rat IgG2aκ), CD25-RPE (rat IgGI), CD11c-biotin-labeled (clone HL3; hamster IgG1), CD11b-FITC (rat IgG2b), CD45R-FITC (clone B220; rat IgG2a), murine FcγRII/III-RPE (clone 2.4G2; rat IgG2b), GR-I-RPE (clone RB6-8C5; rat IgG2b), Streptavedine-RPE, CD80 (clone 16-10A1; hamster IgG2), and CD86 (clone GL1; rat IgG2) were all purchased from PharMingen (BD Biosciences, BD PharMingen, San Diego, Calif.). F4/80-FITC (clone Cl: A3-1; rat IgG2b), was obtained from Serotec (Oxford, UK). Monoclonal antibodies M5/114, anti-mouse MHC II was provided by Dr. Georg Kraal (Vrije Universiteit, Amsterdam, the Netherlands), and 4D11 (clone mLGl-1; rat IgG2a) was produced the hybridoma in our own laboratory.³⁶ FITC-conjugated mouse anti-human FcγRI (CD64; mAb 22; murine IgGI), mAb 32.2 (murine IgG1), and unconjugated murine mAb against HER-2/neu (520C9; murine IgG1) were provided by Medarex (Medarex Inc., Annandale, N.J.). A number of mAb were used for counter-staining when unconjugated. primary mAb were used, including: F(ab′)2 goat-anti-mouse IgG (H+L) (Protos Immunoresearch, San Francisco Calif.), F(ab′)2 donkey-anti-rat IgG (H+L)-FITC, and F(ab′)2 goat-anti-human IgG-FITC (Jackson ImmunoResearch, West Grace, Pa.). c-neu (Ab5; clone TA-l; murine IgG1) was used to assess HER-2/neu expression levels on tumor cell lines (Oncogene, Cambridge, England).³¹ Murine IgG1 and IgG2a anti-idiotype mAb (clone MS5A10 and MS11G6) have been described previously and were purified from cell culture supernatant by affinity chromatography using protein A.^{38,39} BsAb MDX-H210 (hFcγRI×HER-2/neu) was produced by chemically cross-linking F(ab′) fragments of the target antibodies H22 (hFcγRI), and 520C9 (HER-2/neu) (Medarex).⁴⁰

CpG ODN: CpG ODN were provided by Coley Pharmaceutical Group (Wellesley, Mass.). CpG ODN 1826 (sequence TCCATGACGTTCCTGACGTT), was used as the immunostimulatory CpG ODN and CpG ODN 1982 (sequence TCCAGGACTTCTCTCAGGTT), in which the CpG motifs were mutated, served as its control. CpG ODN were tested and proved to contain<12.5 ng/mg of lipopolysaccharides levels by Limulus assays (LAL-assay BioWhittaker, Walkersville, Md.).

Flow cytometry: Whole murine blood, isolated human or murine white blood cells, or murine tumor cells were incubated for 30 minutes with either labeled or unlabeled mAb. Subsequently, cells were washed thrice in PBS supplemented with 1% bovine serum albumin (BSA) and 0.01% azide. When unlabeled primary mAb were used, counter-staining was performed with FITC- or RPE- labeled F(ab′)2 fragments of a goat anti-mouse antibody. Whole blood samples were lysed and fixed by using FACS® lysing solution (Becton Dickinson, San Jose, Calif.). All samples were analyzed on a FA CSCalibur flow cytometer (Becton Dickinson).

Cytotoxicity assay: Tumor cells were plated in 6 well plates at a concentration of 5×10³ cells/well and CpG ODN were added at different concentrations (2.8, 5, 7.5, 10, and 20 μg/ml). Plates were incubated at 37° C. in a humidified incubator and cells were inspected daily by microscopy for proliferation and morphology. At day four, cells were detached with trypsin-EDTA and tested for viability and HER-2/neu expression by FACS analyses. Cells were stained with unconjugated anti-HER-2/neu mAb c-neu (1:20) and counter-stained with F(ab′)2 goat-anti-mouse IgG (H+L) FITC (1:100). After initial analysis of the samples, they were measured a second time following the addition of 10 μl of propidium iodine (1 mg/ml; 1:100), to quantify the percentage of dead cells.

ADCC assay: A Chromium-51 (⁵¹Cr) release assay, slightly modified from Valerius et al., was used.²⁴ Briefly, tumor cells were incubated with 200 μCi ⁵¹Cr (Amersham, Buckinghamshire, UK) for two hours. After washing thrice with culture medium, 5×10³ target cell were added to round-bottomed microtiter plates containing sensitizing mAb/BsAb, CpG ODN, and 50 μl of whole blood. Murine whole blood was used as the source of effector cells. Mice were treated for three days with 150 μg of murine granulocyte colony-stimulating factor (G-CSF) subcutaneous (s.c.) (Amgen, Thousand Oaks, Calif.), to increase circulating numbers of leukocytes prior to blood collection via orbital puncture. The effector to target cell ratio was approximately 80:1 in a final volume of 200 μl. Following overnight incubation at 37° C., assays were stopped by centrifugation. ⁵¹Cr-release was measured in supernatants from triplicate wells. Percentage of cellular cytotoxicity was calculated using the formula:

Experimental cpm−basal cpm/% specific lysis=maximal cpm−basal cpm×100

with maximal ⁵¹Cr release determined by adding Zap-oglobin® (Coulter Electronics LTD. Luton, England; 10% final concentration) to target cells and basal release measured in the absence of sensitizing antibodies and effector cells. Only very low levels of antibody- mediated non-cellular cytotoxicity (without effector cells) and antibody independent killing were observed under these conditions (<5% specific lysis).

Tumor models: In the 38C13 murine B cell lymphoma model, 5×10³ 38C13 T3C tumor cells, which grow rapidly and consistently in immunocompetent C3H/HeN mice, were injected intraperitoneally (i.p.) and CpG ODN and mAb were injected according to the study protocol. A s.c. solid tumor, documented in detail elsewhere³², was established by inoculating 2×10⁶ CMS7HE cells s.c. in the right flank of male F12 Tg-hFcγRl and NTg mice. These tumors grew uniform and could easily be measured using calipers. Tumor volume was reported as length×width×height (mm³). BsAb were injected twice-daily i.p. and CpG ODN s.c. in the vicinity of the tumor or in the neck according to the study protocol. Tumor cells were tested for stable HER-2/neu expression after in vivo passage by FACS analyses. Mice were checked three times a week for signs of toxicity and discomfort, including level of activity, ruffled fur, diarrhea, and general appearance.

Statistical analyses: Group data were reported as mean±standard error of the mean (SEM). Differences between groups were analyzed by unpaired (or, when appropriate, paired) Student's t-tests. Levels of significance are indicated, with significance accepted at the p<0.05 level.

### Example 1

### CpG ODN Enhances the Efficacy of Anti-Tumor mAb

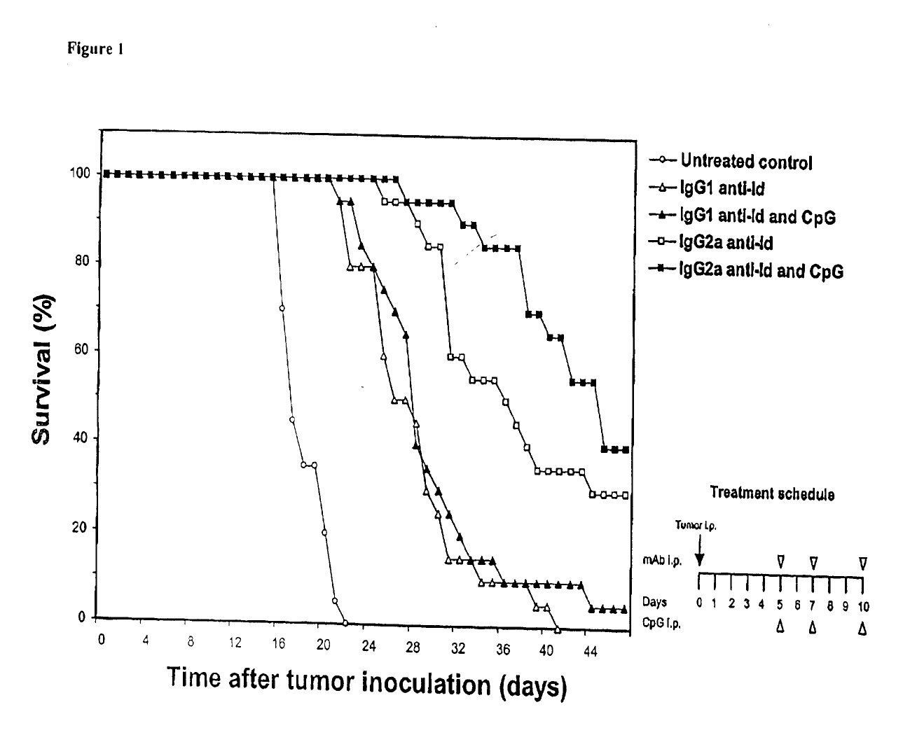

Previous studies in the 38C13 murine B cell lymphoma model have demonstrated that anti-idiotype murine mAb of the IgG2a and IgG1 isotypes exhibited anti-tumor activity with IgG2a mAb being more effective.³⁴ CpG ODN enhanced the efficacy of anti-tumor IgG2a antibodies in this model.⁸ The effect of CpG ODN on the anti-tumor activity of the MS5A1O IgG1 mAb was assessed. Treatment with an IgG2a antibody of the same specificity was also evaluated. CpG ODN enhanced the efficacy of the IgG2a mAb. In contrast, we observed no detectable effect on the efficacy of the IgG1 mAb (FIG. 1).

### Example 2

### Induction of PMN hFcγRI Expression by CpG ODN

One major difference between murine IgG1 and IgG2a mAb is the ability of the IgG2a mAb to bind to hFcγRI.^{28,29} Therefore, the effect of CpG ODN on this receptor in a hFcγRI transgenic murine model was evaluated. hFcγRI Tg FVB/N mice constitutively express hFcγRI on monocytes, macrophages, immature dendritic cells (DC), and in low numbers on polymorphonuclear cells (PMN).³⁰ Expression of hFcγRI on PMN can be upregulated in vivo upon stimulation with IFN-γ or G-CSp.^{41,42} F12 mice showed identical expression patterns and hFcγRI regulation. In contrast to most other leukocyte populations, little is known about the effect of CpG ODN on PMN.

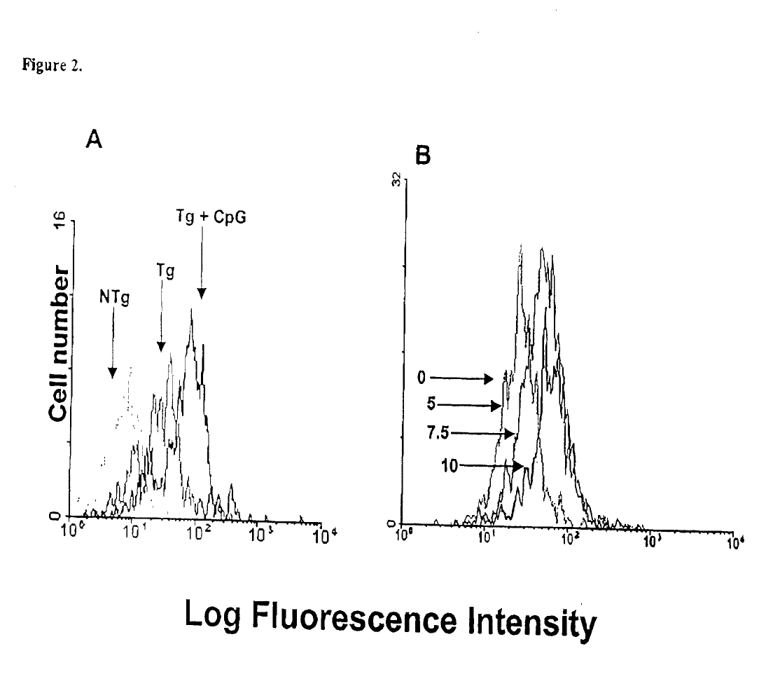

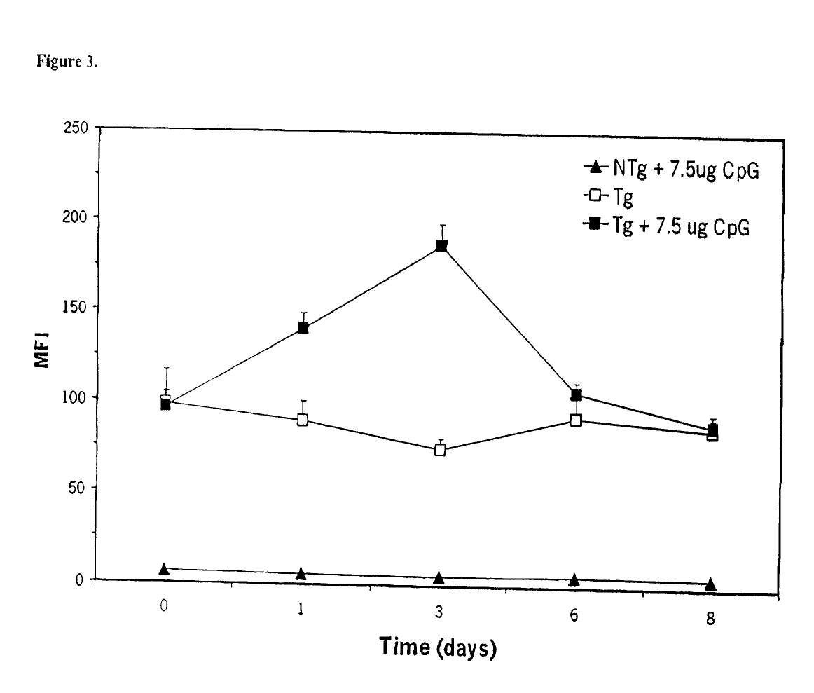

Accordingly, it was tested whether CpG ODN altered hFcγRl expression on PMN in vivo. Three days after a single s.c. dose of CpG ODN 1826, hFcγRI expression was determined. As illustrated in FIG. 2A, hFcγRI was expressed on PMN of Tg mice and an enhanced expression was seen in Tg mice treated with CpG ODN 1826. Conversely, no hFcγRI expression was detected in NTg mice. Treatment with CpG ODN 1826 also enhanced hFcγRI expression levels on monocytes and DC (n=3, data not shown). In addition, hFcγRI expression levels were upregulated by CpG ODN in a dose-dependent manner (FIG. 2B). The kinetics of hFcγRI expression after a single s.c. dose of CpG ODN 1826 was assessed. A clear time-response curve was observed, with upregulation of hFcγRI expression for up to 5 days after a single dose of CpG ODN 1826 (FIG. 3).

### Example 3

### Immunostimulatory Effects of CpG ODN on Phagocytic Cells

To assess whether CpG ODN 1826 was immunostimulatory for phagocytic cells, hFcγRI Tg and NTg mice were injected with a single dose of CpG ODN 1826 at day 0. At different time points, 100 μl of whole blood were obtained and PMN, monocyte, DC, T cell, and B cell populations were analyzed by flow cytometry.

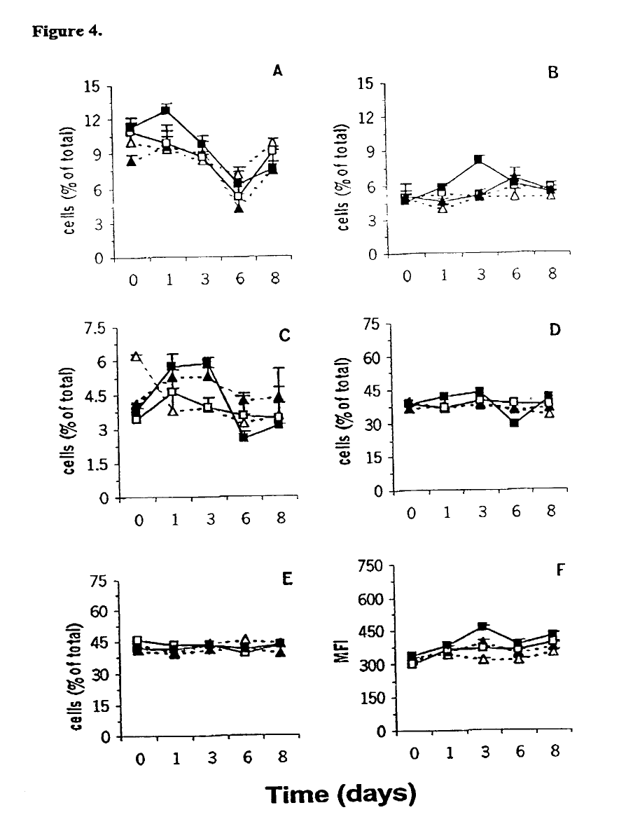

A clear increase was observed in the percentage of PMN at day 1, which then returned to baseline by day 3. Monocytes showed a comparable pattern, with maximal cell numbers reached at day 3. For DC, both Tg and NTg mice showed an increase, although to a different extent. There was no change in B cell and T cells numbers (FIGS. 4A-E).

In addition to the lineage-specific markers, activation markers (MHC II, B7-1, B7-2 were also studied, and murine FcγRII/III). FcγRII/III expression assessed in the Tg animals showed a slight increase (in MFI) on day 3 (FIG. 4F). No changes were observed in any of the other activation markers.

### Example 4

### Effect of CpG ODN on hFcγRI-Mediated ADCC

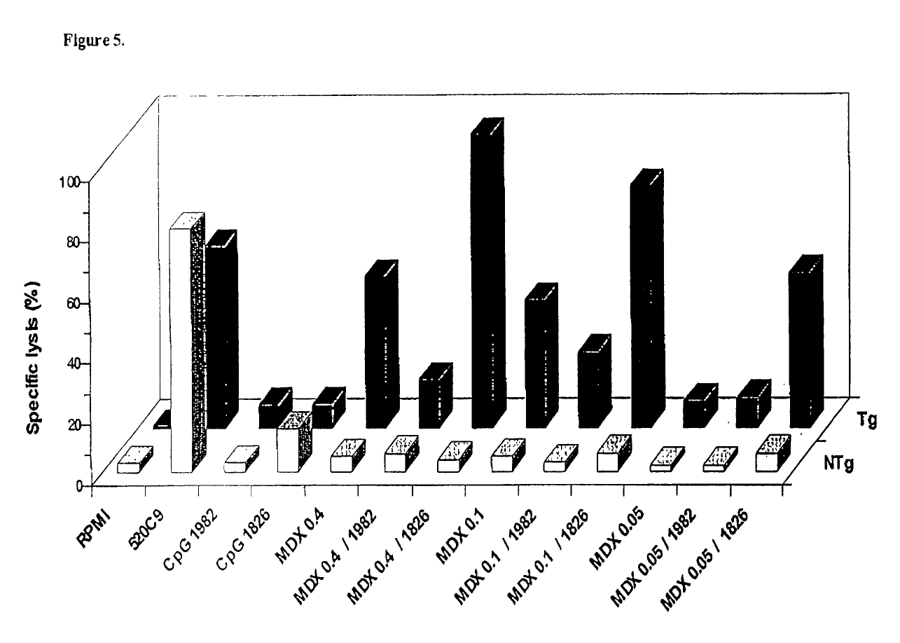

MDX-H210 is a bispecific antibody (BsAb) directed against hFcγRI (CD64) and the tumor antigen, HER2/neu. It was investigated whether MDX-H210-mediated ADCC by G-CSF stimulated murine PMN was affected by CpG ODN 1826. Transgenic PMN exhibited enhanced tumor cell killing via MDX-H210 following addition of CpG ODN 1826. Furthermore, the combination of MDX-H210 and CpG ODN 1826 was effective at very low BsAb concentrations (FIG. 5). No enhanced specific lysis was observed with a control CpG ODN 1982 in combination with MDX-H210. NTg PMN were unable to mediate lysis except via mAb 520C9, an anti-HER-2/neu murine IgG1 mAb that initiates cytotoxicity via murine FcγRII/III.⁴³

### Example 5

### Effect of CpG ODN on BsAb-Induced Anti-Tumor Activity

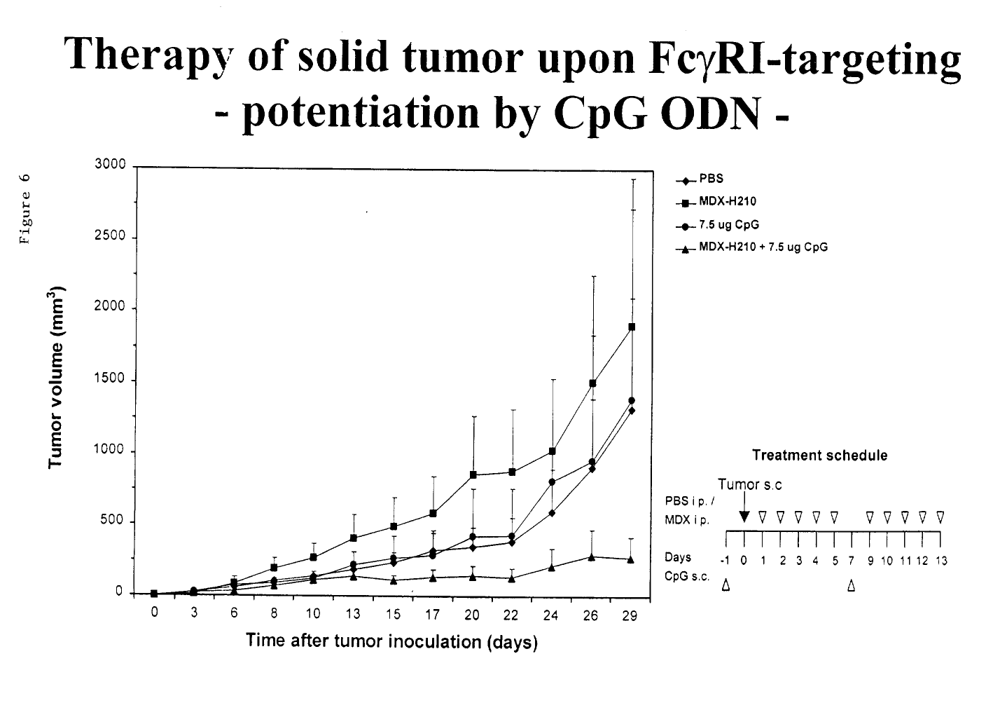

To evaluate whether CpG ODN alone were cytotoxic for solid tumor cells, several cell lines SK-BR-3, RZ#14+, CMS7neo, and CMS7HE were tested. CpG ODN 1826 and 1982 did not affect cell morphology, proliferation, antigen expression, or viability (n=3, data not shown). Since CpG ODN did not exhibit a direct toxic effect on tumor cells, but did induce immune stimulation of phagocytic cells resulting in enhanced hFcγRI expression levels, their effect in vivo was further assessed. The capacity of hFcγRI to trigger anti-tumor effects by using a hFcγRI-directed BsAb in combination with CpG ODN was tested. Mice bearing a murine fibrosarcoma (CMS7HE) were treated with a combination of MDX-H210 and CpG ODN 1826. A clear reduction in the growth of tumors was observed in Tg mice treated with the combination of MDX-H210 and CpG ODN 1826, whereas in all other treatment groups (and in NTg mice) tumors grew progressively (FIG. 6). Experiments performed with inactive CpG ODN 1982 did not show any therapeutic effects.

Conclusion

Examples 1-5 show that CpG ODN increase expression of FcγRI. A single s.c. injection of CpG ODN upregulated hFcγRI expression on murine PMN in a time and concentration-dependent manner. In addition, absolute numbers of PMN, monocytes, and DC, were increased with no apparent changes in T- and B cell numbers. CpG ODN are known to induce a Th1- instead of a Th2-type response. This results in a cytokine profile which favors hFcγRI-upregulation.⁴⁶ Earlier studies by Valerius et al.^{24,47} documents upregulation of hFcγRI to result in highly functional phagocytic cells. The studies described in Examples 1-5 show that CpG ODN not only enhances the expression of hFcγRI, but also the phagocytic capacity of hFcγRI-positive cells. Specifically, increased cytotoxicity was observed using hFcγRI-positive PMN in ADCC via an hFcγRI-directed BsAb in combination with CPG ODN.

It can also be concluded from the studies described in Examples 1-5 that CpG ODN enhance IgG2a-induced anti-tumor effects. Based on the results observed, and the specificity of murine IgG2a mAb for FcγRI^{28,29}, the studies confirmed that FcγRI plays a central role in the ability of CpG ODN to enhance the efficacy of mAb therapy, and that synergy exists between CpG ODN and hFcγRI-mediated lysis in vivo.

The data obtained in the solid tumor model studies described in Examples 1-5 show the role of hFcγRI in the enhancement of anti-tumor effects following CpG ODN administration in vivo. CpG ODN clearly enhanced MDX-H210-mediated growth inhibition and ADCC of tumor cells expressing HER2/neu positive tumor cells. The results observed also showed that hFcγRI positive PMN, monocytes, and macrophages play an important role in mediating the anti-tumor activity of CpG ODN when administered together with anti-tumor mAb.

Accordingly, Examples 1-5 demonstrate that the CpG ODN compositions of the present invention induce both direct anti-tumor effects and active anti-tumor immune responses. Enhancement of both ADCC and the development of active anti-tumor immune responses was accomplished through the use of CpG ODN in combination with hFcγRI-directed approaches.

Part II—Immunostimulatory Oligonucleotides Enhance FcR (CD64)-Mediated Antigen Presentation

Materials and Methods

Mice: Human CD64 Tg animals⁸ crossed with C57B16 (F1) or Balb/c mice (F12) were bred and maintained at the Transgenic Mouse Facility of the Central Animal Laboratory, Utrecht University, the Netherlands. C57B16 and Balb/c mice were obtained from Harlan (Horst, The Netherlands). 8-12 wk old human CD64-expressing animals were used in the experiments, as well as their NTg littermates. All experiments were approved by the Utrecht University animal ethics committee.

Cell lines: The RF33 cell line, expressing a TCR recognizing the H-2^{b}-restricted OVA epitope SIINFEKL⁴⁴, and the OVA-specific D011.10 cell line, that recognizes the OVA peptide in an Ia^{d} restricted way⁴⁵, were cultured in RPMI 1640 medium (Gibco BRL, Life Technologies, Paisley, Scotland), supplemented with 10% heat-inactivated fetal bovine serum (FBS) (Fetalclone I, Hyclone, Logan, Utah), 50 IU/ml penicillin (Gibco BRL) and 50 μg/ml streptomycin (Gibco BRL). The interleukin 2 (IL-2) dependent CTLL-2 cell line⁴⁶ was propagated in RPMI 1640 culture medium, with 10% FBS, 50 IU/ml penicillin, 50 μg/ml streptomycin and 100 U/ml IL-2 (Immunokine, Boehringer Ingelheim, Alkmaar, The Netherlands).

Antibodies: CD80 (clone 16-10A1), CD86 (clone GL1), CD11c biotin (clone HL3), CD32/16 PE (clone 2.4G2), Gr-1 PE (clone RB6-8C5), CD45R/B220 biotin (clone RA3-6B2), CD3 FITC (clone 17A2) were obtained from PharMingen (BD Biosciences, BD PharMingen, San Diego, Calif.). CD64 PE and SA-PE were purchased from Becton Dickinson (BD Biosciences, San Jose, Calif.). F4/80 biotin (clone Cl: A3-1) was obtained from Serotec (Oxford, UK). CD40 PE (clone 3.23) was purchased from Immunotech (Marseille, France), and F(ab′)₂ fragment mouse anti-rat IgG (H+L) was purchased from Jackson Immunoresearch (West Grace, Pa.). NLDC-145⁴⁷ and M5/114 anti-class II⁴⁸ were kindly provided by Dr. Georg Kraal (Vrije Universiteit, Amsterdam, The Netherlands).

Antigens: The immunodominant peptide of OVA, SIINFEKL (OVA 257-264), was obtained from Isogen (Maarssen, The Netherlands). Ovalbumin complexes were generated by incubation of 40 μg/ml chicken egg OVA (Sigma, St.Louis, Mo.) with 80 μg/ml specific rabbit IgG serum (Sigma) for 20 min at 37° C. 22×OVA conjugates were prepared using N-succinimidyl S-acetylthioactetate (SATA) (Pierce, Rockford, Ill.) and SPDP (Pierce) as chemical cross-linkers.⁴⁹ 22-OVA fusion protein was generated as follows. The V_{H} and V_{L} encoding regions of anti-CD64 monoclonal antibody H22 were obtained by PCR. The primers (purchased from GenoSys Biotechnologies, The Woodlands, Tex.) for V_{H} were GATCGATCGATATCCAACTGGTGGAGAGCGGTG for the forward primer and

GTACTCAGTCCGGAGCCGCCACCTCCTGAGCTCACGGTGACCGGGGTCCCTTG for the reverse primer. The primers for V_{L} were

GTACTCAGTCCGGAGGTGGAGG

CAGCGGAGGGGGCGGATCCGACATCCAGCTGACCCAG for the forward primer and

CAGTCAGTTCTAGAGTCAGCTCGAGCAGCTAGATTTGATTTCCACCTTGGTCC for the reverse primer. An expression vector encoding H22 light chain was used as template. PCR was carried out using the GeneAmp PCR Reagent Kit with Amplitaq DNA Polymerase (PE Applied Biosystems, Foster City, Calif.) according to the manufacturer's instructions. Amplified DNA fragments were purified and then ligated separately into the vector pcDNA3/CAT (Invitrogen, Carlsbad, Calif.). DNA sequencing was done by National Biosciences, Inc. (Plymouth, Minn.) to confirm the integrity of the V_{H} and V_{L} genes. An immunoglobulin signal sequence (MGWSCIILFLVATATGVHS) was constructed by annealing two complimentary oligomers encoding the signal sequence and ribosome-binding site. The sense oligomer was

AGCTTCACCATGGGATGGAGCTGTATCATCCTCTTCTTGGTGGCCACAGCTA CCGGTGTCCACTCCGAT and the antisense oligomer was ATCGGAGTGGA CACCGGTAGCTGTGGCCACCAAGAAGAGGATGATACAGCTCCATCCCATGG TGA. The last steps were to construct a coding region at the 3′-end of the sFv, to add a small linker (Gly4Ser), a c-myc tag (EQKLISEEDLN), and a 6-His tail. This gene fragment was constructed using 4 oligomers:

A=3DTCGAGCGGAGGCGGGGGTAGGGATATCGCGGCCGCAGAACAGAAACT C,

B=3DTGAGATGAGTTTCTGTTCTGCGGCCGCGATATCGCTACCCCCGCCTCCG C,

C=3DATCTCAGAAGAGGATCTGAATGGCGCCGCACATCACCATCATCACCAT TGATT,

D=3DCTAGAATCAATGGTGATGATGGTGATGTGCGGCGCCATTCAGATCCTC TTC. The forward primer: ATAAGAATGCGGCCGCAGGCTCCATCGGCGCAGC and the reverse primer: ATAAGAATGCGGCCGCAGGGGAAACACATCTGC were used to perform PCR on the cDNA encoding the OVA sequence. The product was then digested and inserted into the 22 sFv containing pJG225 plasmid, producing a 22 sFv and OVA gene fusion (22-OVA). The correct orientation, frame and integrity of this gene fusion were confirmed by DNA sequencing (Molecular Biology Core Facility, Dartmouth Medical School, Hanover, N.H.). To generate 22-OVA recombinant baculovirus, the 22-OVA sequence was inserted into the pVL1393 baculovirus transfer vector (BD PharMingen, San Diego, Calif.), and Spodoptera frugiperda (Sf9) insect cells (BD PharMingen) were co-transfected with linearized baculovirus DNA and the 22-OVA pVL1393 vector using the BD Baculogold Transfection kit as recommended by the manufacturer. Trichopluscia Ni (Hi-5) insect cells (Invitrogen) were infected with high titer baculovirus encoding 22-OVA at a multiplicity of infection of 10. After four days, the supernatant was collected and concentrated and the protein construct was purified using a protein L column (Clontech, Palo Alto, Calif.). Purified protein constructs were run out on 6% acrylamide gels and stained with Coomassie brilliant blue to test for purity. In addition, all protein constructs were tested for lipopolysaccharide (LPS) contamination by the Limulus Amebocyte Lysate QCL-1000 assay kit (BioWhittaker, Walkersville, Md.).

CpG ODN: Synthetic ODN were provided by Coley Pharmaceutical Corporation (Wellesley, Mass.). CpG ODN 1826 with the following sequence was used: TCCATGACGTTCCTGACGTT. In addition, CpG ODN were tested for LPS contamination by the Limulus Amebocyte Lysate QCL-1000 assay kit (BioWhittaker, Walkersville, Md.).

Generation of DC: Bone-marrow derived DC (BMDC) were obtained as described by Inaba.⁵⁰ Briefly, bone marrow was flushed from mouse femurs, erythrocytes were lysed and cells were grown at 1×10⁶/ml in filtered RPMI⁺ (RPMI 1640 medium with 10% FBS, 50 IU/ml penicillin and 50 μg/ml streptomycin) in the presence of either 10 ng/ml granulocyte/macrophage colony-stimulating factor (GM-CSF; Immunex, Seattle, Wash.) or 10 ng/ml GM-CSF+50 ng/ml tumor necrosis factor alpha (TNF-α; Hycult, Uden, The Netherlands). Non-adherent cells were replated on day 1, and non-adherent cells were removed on days 2 and 4 from the cultures, with concomitant refreshment of culture media. Non-adherent and loosely adherent DC were harvested on days 7, 8, or 9.

Flow cytometric analyses: Day 7 DC (DC7) (1.105), day 8 DC cultured for 24 h with 100 μg/ml CpG ODN and day 9 DC (DC9), part of which were cultured for 48 h with 1 μg/ml LPS (Sigma), were blocked with 5% heat-inactivated mouse serum for 30 min at room temperature (RT). Cells were washed with FACS buffer (phosphate-buffered saline (PBS), 0.1% azide, 1% bovine serum albumin (BSA)) and incubated with relevant antibodies for 20 min at RT. Cells were washed and, if required, incubated with a specific secondary antibody. Cells were analyzed by flow cytometry using a FACSCalibur (BD Biosciences). Unstained cells, isotype controls, and secondary (fluorochrome-labeled) antibodies were used as negative controls.

MHC class II antigen presentation assay: DC7 or DC9 (1×10⁵ cells) were washed twice in RPMI⁺ and resuspended in 100 μl RPMI⁺/well. DC were incubated with various concentrations of OVA-IgGαOVA complexes and 1×10⁵ DO11.10 T cells for 24 h at 37° C. An excess of OVA (0.4 mg/ml) was used as a positive control. The presence of IL-2 released by the D011.10 cells was determined by culturing 5×10³ IL-2 dependent CTLL-2 cells with various culture supernatants. After overnight incubation, 1 μCi of ³H Thymidine (Amersham, Buckinghamshire, UK) was added to each well, and cells were harvested 24 h later onto glass fibre filters (Wallac, Turku, Finland) for liquid scintillation counting.

MHC class I antigen presentation: DC7 (1×10⁵) were washed twice in RPMI⁺ medium and resuspended in 150 μl RPMI⁺/well. DC were incubated with OVA-IgGαOVA complexes, 22-OVA or 22×OVA, either with or without 10 μg/ml CpG ODN 1826 for 24 h at 37° C. The SIINFEKL peptide (0.29 mg/ml) served as a positive control. After 24 h, cells were washed once with RPMI⁻ medium (RPMI 1640 medium only) and fixed using 1.5% paraformaldehyde for 20 min at RT. Cells were washed once in RPMI⁻ medium and quenched with 50 mM NH₄CL for 60 min at RT. Cells were washed three times in RPMI⁻ medium and resuspended in 100 μl RPMI⁺/well. RF33 cells, 1×10⁵, were added in a volume of 50 μl, and incubated for 36 h at 37° C. Hundred μl culture supernatant were harvested from each well. The presence of IL-2 released by the RF33 cells in culture supernatants was determined as above.

### Example 6

### Effect of Cell Culture on Dendritic Cell (DC) Phenotype

Murine DC can be obtained in large numbers by culturing bone marrow cells with growth factors such as GM-CSF, GM-CSF/IL-4, or GM-CSF/TNF-α. Time of culture and specific types of growth factors used are critical for the maturation state and FcR expression profiles of DC.^{50,51,52} Immature DC, which are efficient in (receptor-mediated) uptake of antigens, are characterized by low/intermediate expression of co-stimulatory- and MHC class II molecules. When DC mature, co-stimulatory and MHC class II molecules are up-regulated, with concomitant down regulation of the capacity of receptor-mediated uptake and processing of exogenous antigens.²⁵

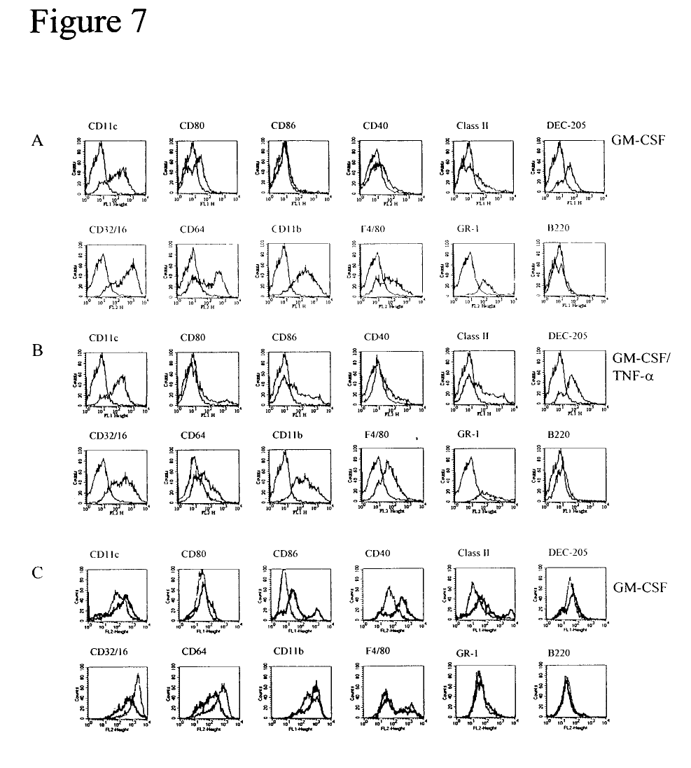

To assess the maturation state and FcR expression pattern of CD64-Tg and NTg DC cultured with GM-CSF, GM-CSF/IL-4, or GM-CSF/TNF-α, cell surface marker expression was analyzed by flow cytometry. After a culture period of 7 days, non-adherent and loosely adherent cells (DC7) were stained for expression of DC markers (i.e., CD11c, and DEC-205), co-stimulatory molecules (i.e. CD40, CD80, and CD86), MHC class II, and FcR. Although GM-CSF/IL-4 cultured DC exhibited a typical DC phenotype, human CD64 expression was low (n=3, data not shown). This is likely attributable to the fact that IL-4 down-regulates CD64 expression on myeloid cells.^{32,53} Culture protocols using GM-CSF or GM-CSF/TNF-α resulted in DC with a characteristic DC phenotype, as shown by expression of CD11c, and DEC-205. DC7 exhibited an immature phenotype with low/intermediate CD86 and CD80 expression. However, GM-CSF/TNF-α DC7 expressed CD86 and MHC class II at higher levels than GM-CSF DC7, indicating a more mature phenotype. In addition, DC7 was found to express mycloid markers such as GR-1 and F4/80, and no B cell (FIGS. 7A, B) or T cell markers (n=4, data not shown). Both GM-CSF and GM-CSF/TNF-α DC7 expressed human CD64. CD64 expression was lower on GM-CSF/TNF-α DC7 (FIGS. 7A,B). This is consistent with the finding that mature human DC down-regulate CD64 expression.⁵⁴ When DC were cultured for 9 days (DC9), both culture protocols resulted in a more mature DC phenotype, as reflected by up regulation of co-stimulatory and MHC class II molecules.

Immature DC can be triggered to develop into mature DC by inflammatory stimuli such as LPS.⁵⁵ The effect of co-culturing human CD64-Tg and NTg GM-CSF and GM-CSF/TNF-α DC7 with LPS for two days was tested. Both GM-CSF (FIG. 7C) and GM-CSF/TNF-α DC showed maturation as reflected by up regulation of co-stimulatory molecules (such as CD40, CD80 and CD86), MHC class II, and DC markers (i.e. CD11c and DEC-205). Human CD64 expression was found to be down-regulated upon maturation.

### Example 7

### MHC Class II Antigen Presentation Capacity

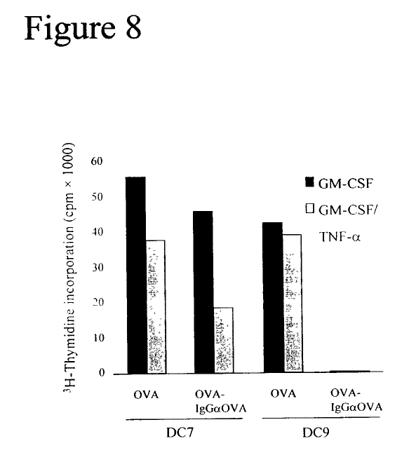

As DC maturation state is characterized not only by phenotype but also by function, the MHC class II antigen-presenting capacity of DC7 and DC9 were tested. DC were either incubated with excess OVA (400 μg/ml), to assess the capacity of fluid phase antigen uptake and subsequent processing, or with 100 ng/ml OVA-IgGαOVA immune complexes, to study FcR-mediated uptake/processing. As a read out for OVA antigen presentation, OVA-specific MHC class II-restricted D011.10 T cells were used. When OVA was administered at high concentrations, both human CD64-Tg (FIG. 8) and NTg (n=4, data not shown,) DC7 and DC9 were capable of fluid phase-triggered internalization and processing of OVA. However, levels of FcR-mediated OVA uptake, and subsequent MHC class II presentation were significantly lower in DC9. This indicated that a more mature DC phenotype (upon culture for 9 days) resulted in down-regulated receptor-mediated uptake and processing.

Although culturing with GM-CSF/TNF-α resulted in higher MHC class II expression levels (FIG. 7), GM-CSF DC7 were more effective in MHC class II antigen presentation, than GM-CSF/TNF-α DC7 (FIG. 8). This may be attributable to the fact that TNF-α triggers activation and functional maturation of DC⁵¹, which was reflected in less efficient FcR-mediated MHC class TI antigen presentation. GM-CSF and GM-CSF/TNF-α DC7 were used to study human CD64-mediated cross presentation in detail.

### Example 8

### Effect of CpG ODN on DC Cell Surface Markers

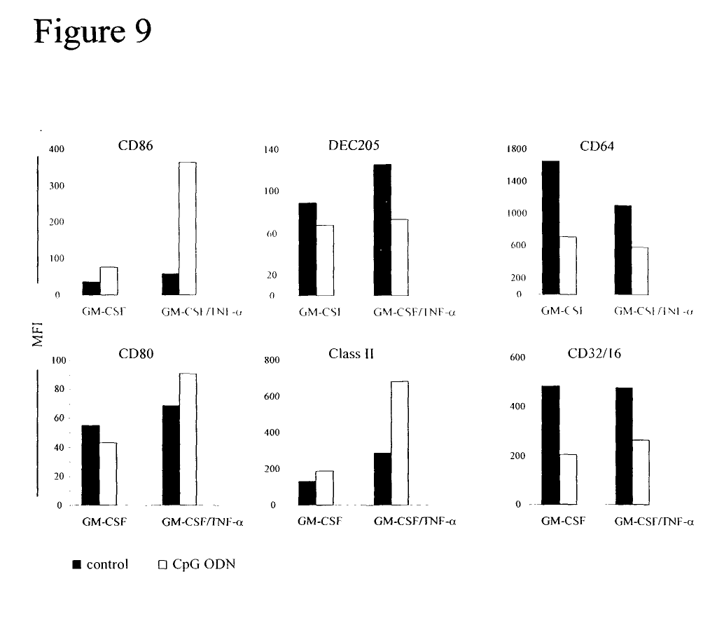

CpG ODN can exhibit direct effects on DC differentiation and maturation and enhance cross presentation of MHC class I-restricted peptides and fluid phase-internalized antigens.⁴¹ To study the effect of CpG ODN on FcR-triggered antigen presentation, the effect of CpG ODN on cell surface marker expression of GM-CSF and GM-CSF/TNF-α immature DC was analyzed. DC7 were incubated with CpG ODN for 24 h and cell surface marker expression levels were examined. CpG ODN activated DC7 as reflected by up regulation of co-stimulatory and MHC class II molecules and down regulation of FcR, such as human CD64, and mouse CD32/CD16 (FIG. 9). The DEC-205 molecule was down-regulated on DC by CpG ODN (FIG. 9), whereas LPS led to its up regulation (FIG. 7C). This finding may be attributable to the fact that DC are differentially sensitive to CpG ODN and LPS.⁴¹

CpG ODN stimulation had much greater effects on co-stimulatory and MHC class II marker expression of GM-CSF/TNF-α DC compared to GM-CSF DC (FIG. 9).

### Example 9

### CpG ODN Enhance Cross Presentation of FcR-Targeted Immune Complexes

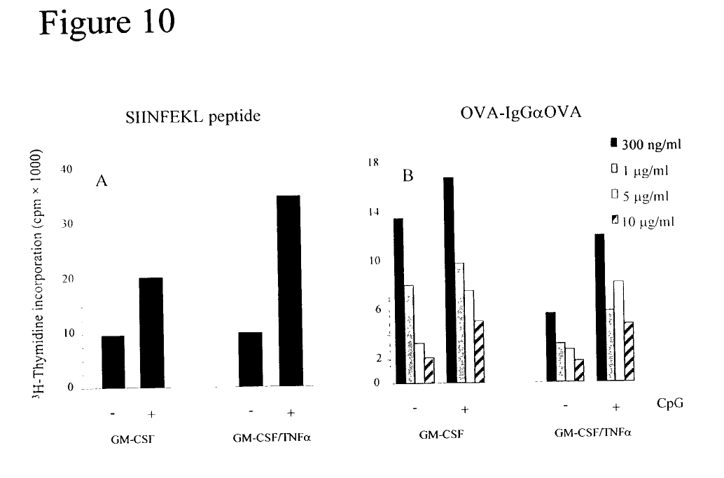

The adjuvant effect of CpG ODN on FcR-triggered cross presentation were studied. Human CD64-Tg and NTg GM-CSF or GM-CSF/TNF-α DC7 were incubated with different concentrations of OVA-IgGαOVA immune complexes, either with or without CpG ODN. After 24 h, DC were fixed and MHC class I-restricted OVA-specific RF33 T cells were added for 36 h. IL-2 release was assessed using CTLL-2 proliferation assays. The immunodominant OVA peptide SIINFEKL served as a positive control for MHC class I-restricted antigen-presenting capacity. Upon incubation with peptide, human CD64-Tg GM-CSF and GM-CSF/TNF-α (FIG. 10A) and NTg, DC7 showed similar ³H-thymidine incorporation levels. In addition, CpG ODN stimulation of these DC clearly enhanced peptide presentation (FIG. 10A). Both human CD64-Tg GM-CSF and GM-CSF/TNF-α DC7 (FIG. 10B) and NTg DC7 were found efficient in cross presentation of OVA immune complexes at low concentrations. This process was enhanced two- to four-fold when DC were stimulated with CpG ODN. Marker analyses showed CpG ODN-stimulated GM-CSF/TNF-α DC7 to express higher levels of co-stimulatory molecules (FIG. 9). Furthermore CpG ODN stimulation of GM-CSF/TNF-α DC7 resulted in a more efficient MHC class I presentation of the OVA peptide. However, FcR-mediated MHC class I cross presentation of immune complexes was lower, compared to GM-CSF DC7 (FIG. 10).

### Example 10

### CpG ODN Enhance Cross Presentation of Human CD64-Targeted Antigens

The capacity of human CD64 to induce DC-mediated cross presentation of OVA and the effect of CpG ODN stimulation on this process was studied as follows.

GM-CSF and GM-CSF/TNF-α DC7 were generated from bone marrow of human CD64-Tg mice and NTg littermates. To target OVA to human CD64, we followed two approaches were followed. First, a sFv fragment of monoclonal antibody H22 to human CD64 was genetically linked to OVA (22-OVA). This molecule targets OVA to human CD64, without any cross-linking of the receptor. Second, OVA was chemically cross-linked to whole IgG of monoclonal antibody H22 (22×OVA) which results in a molecule targeting OVA to human CD64 that cross-links the receptor. DC7 were incubated with either 22×OVA, or 22-OVA, with or without CpG ODN for 24 h. DC were fixed and subsequently incubated with MHC class I-restricted OVA-specific RF33 T cells for 36 h. Levels of IL-2 production by RF33 T cells were determined by CTLL proliferation.

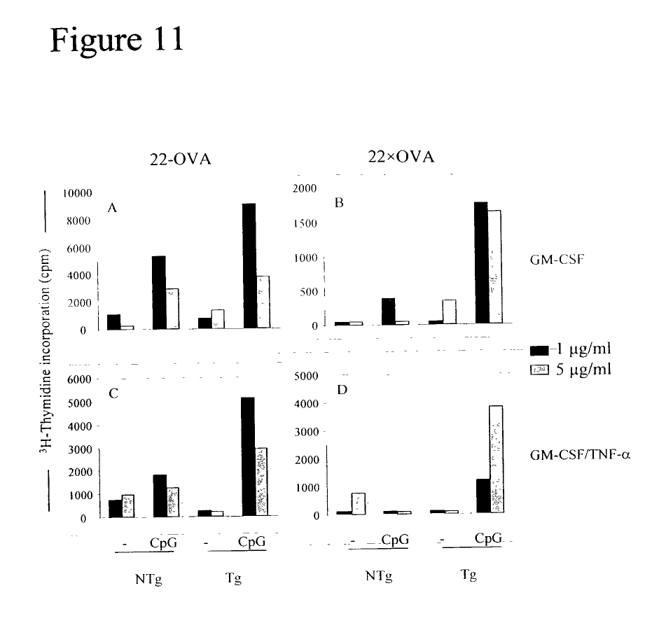

No human CD64-triggered cross presentation of OVA was observed with either Tg GM-CSF or GM-CSF/TNF-α DC7. However, when CpG ODN were added, a significant increase in human CD64-mediated antigen presentation was observed, as reflected by higher antigen-presenting capacities of CpG ODN-stimulated human CD64 Tg DC7, compared to NTg DC7 (FIG. 11). The fact, that both the 22-OVA and 22×OVA triggered cross presentation indicated cross-linking of human CD64 not to be crucial for this process. Furthermore, although GM-CSF/TNF-α expressed CD64 at lower levels and had a more mature phenotype, human CD64-Tg GM-CSF and GM-CSF/TNF-α DC were similarly active in OVA presentation in the presence of 22-OVA, or 22×OVA (FIG. 11). These findings showed that antigens targeted specifically to human CD64 initiated efficient cross presentation by both immature and more mature DC, in contrast to antigens targeted to other (murine) FcR.³⁰

Conclusion

The studies described in Examples 6-10 show that human CD64 is capable of triggering cross presentation by DC, and that CpG ODN enhance this cross-presentation. When co-cultured with DC, CpG ODN enhanced expression of costimulatory molecules, but triggered down regulation of FcR DC expression in vitro. CpG ODN also enhanced MHC class I-restricted peptide presentation, which was positively correlated with a more mature phenotype, as CpG ODN exhibited the largest effect on peptide presentation by GM-CSF/TNF-α DC. This is consistent with data in literature, showing that CpG ODN act as adjuvants for MHC class I-restricted epitopes⁴² and that CpG ODN treatment of peptide- or protein-pulsed DC enhanced the ability of the DC to activate class I-restricted T cells.⁵⁶

FcR-mediated cross presentation of OVA-IgGαOVA immune complexes was two- to four-fold up-regulated upon CpG ODN activation, in spite of the fact that CpG ODN down-regulated FcR expression levels (FIG. 9). FcR-mediated antigen presentation has been reported to be 100- to 1000-fold more efficient, compared to fluid-phase-mediated presentation.³⁰ The results described in Examples 6-10 document for the first time the capacity of CpG ODN to further enhance FcR-mediated cross-presenting processes, in spite of inducing maturation (and concomitant down regulation of FcR). Thus, CpG ODN activation can improve DC-based vaccine therapies.

To study potential in vivo human CD64-targeting strategies, OVA was targeted outside the IgG ligand-binding domain. No human CD64-triggered cross presentation using either GM-CSF of GM-CSF/TNF-α DC was found. However, when DC were activated by CpG ODN, which up-regulated expression of co-stimulatory molecules, enhanced antigen-presenting capacity, but down-regulated human CD64 expression, efficient human CD64-mediated cross presentation was observed. These findings are consistent with earlier data showing that a CD64-targeted antigen co-localizes with MHC class I molecules in human myeloid U937 cells³⁶ and that documented human CD64 to be capable of targeting prostate specific antigen to MHC class I cross-presenting pathways in myeloid THP-1 cells.⁵⁹

In addition, the data obtained from the studies described in Examples 6-10 support the well-documented potent anti-tumor and vaccine responses seen upon targeting of human CD64 in human CD64 transgenic mouse models.^{35,60,61}

Human CD64 exhibits the capacity to efficiently internalize antigens, without a need for massive receptor cross-linking.^{34,62} The data shown in Examples 6-10 also show that such cross-linking appeared also unnecessary for cross presentation. This shows that human CD64 has relatively easy access to the cross-presenting machinery.

Due to the fact that DC represent the most potent initiators of immune responses^{25,40}, and are capable of funneling exogenous antigens through the MHC class I-restricted antigen presentation pathway^{29,41}, they represent important tools for the development of new therapeutic concepts. The data obtained from the studies described in Examples 6-10 confirms that human CD64 is capable of triggering DC cross presentation and that CpG ODN enhance this effect. The data also show that CD64 expression is restricted to mycloid cells (in contrast to other FcR family members)³¹. Accordingly, CpG ODN in combination with CD64-targeting approaches offers a significant improvement over previous DC-based immunotherapies.

Equivalents

Those skilled in the art will recognize, or be able to ascertain using no more than routine experimentation, many equivalents of the specific embodiments of the invention described herein. Such equivalents are intended to be encompassed by the following claims.

References

1. Coley. W. B. The treatment of malignant tumors by repeated inoculations of erysipelas. With a report of ten original cases. 1893. Clin.Orthop., 3-11, 1991

2. Wiemann, B. and Starnes, C. O. Coley's toxins, tumor necrosis factor and cancer research: a historical perspective. Pharmacol. Ther., 64: 529-564, 1994.

3. Smyth, M. J., Godfrey, D. I., and Trapani, J. A- A fresh look at tumor immunosurveillance and immunotherapy. Nat.Immunol., 2: 293-299, 2001

4. Krieg, A. M. and Wagner, H. Causing a commotion in the blood: immunotherapy progresses from bacteria to bacterial DNA. Immunol.Today, 21: 521-526,2000.

5. Tokunaga, T., Yamamoto, H., Shimada, S., Abe, R., Fukuda, T., Fujisawa, Y., Furntani, Y., Yano, O., Kataoka, T., Sudo, T., and Antitumor activity of deoxyribonucleic acid fraction from Mycobacterium bovis BCG. I. Isolation, physicochemical characterization, and antitumor activity. J.Natl.Cancer Inst., 72: 955-962, 1984.

6. Tokunaga, T., Yamamoto, T., and Yamamoto, S. How BCG led to the discovery of immunostimulatory DNA. Jpn.J.Infect.Dis., 52: 1-11, 1999.

7. Krieg, A. M. Immune effects and mechanisms of action of CpG motifs. Vaccine, 19: 618-622,2000.

8. Wooldridge, J. E., Ballas, Z., Krieg, A. M., and Weiner, G. J. Immunostimulatory oligodeoxynucleotides containing CpG motifs enhance the efficacy of monoclonal antibody therapy of lymphoma. Blood. 89: 2994-2998. 1997.

9. Krieg, A. M., Love-Homan, L., Yi, A. K., and Harty, J. T. Cpa DNA induces sustained IL-12 expression in vivo and resistance to Listeria monocytogenes challenge. J.Immlmol., 161: 2428-2434, 1998.

10. Carpentier, A. F., Xie, J., Mokhtari, K., and Delattre, J. Y. Successful treatment of intracranial gliomas in rat by oligodeoxynucleotides containing CpG motifs. Clin.Cancer Res., 6: 2469-2473, 2000.

11. Pegram, M. D., Lipton. A.. Hayes, D. F., Weber, B. L, Baselga, J. M., Tripathy, D., Baly, D., Baughman, S. A, Twaddell, T., Glaspy, J. A., and Slamon, D. J. Phase II study of receptor-enhanced chemosensitivity using recombinant humanized anti-p185HER2/neu monoclonal antibody plus cisplatin in patients with HER2J neu—overexpressing metastatic breast cancer refractory to chemotherapy treatment. J. Clin.Oncol., 16: 2659-2671, 1998.

12. Maloney, D. G. Preclinical and phase I and II trials of rituximab. Semin.Oncol., 26: 74-78, 1999.

13. Baselga, J. Clinical trials of Herceptin(trastuzumab). Eur.J.Cancer, 37 Suppl 1: S18-S24,2001.

14. Eccles, S. A. Monoclonal antibodies targeting cancer: ‘magic bullets’ or just the trigger? Breast Cancer Res., 3: 86-90, 2001

15. Weiner, L. M. An overview of monoclonal antibody therapy of cancer-Semin.Oncol., 26: 41-50, 1999.

16. Cragg, M. S., French, R R, and Glennie, M. J. Signaling antibodies in cancer therapy. Curr.Opin.Immunol., 11: 541-547,1999.

17. Glennie, M. J. and Johnson, P. W. Clinical trials of antibody therapy. Immunol.Today, 21: 403-410, 2000.

18. Clynes, R., Takechi, Y., Moroi, Y., Houghton. A., and Ravetch, J. V. Fc receptors are required in passive and active immunity to melanoma Proc.Natl.Acad.Sci.U.S.A., 95: 652-656.1998.

19. Clynes, R A., Towers, T. L.. Presta, L. G., and Ravetch, J- V. Inhibitory Fc receptors modulate in vivo cytoxicity against tumor targets. Nat.Med., 6: 443-446, 2000.

20. Honeychurch, J., Tutt, A. L., Valerius, T., Heijnen, I. A., van de Winkel, J. G., and Glennie, M. J. Therapeutic efficacy of FcyR1/CD64-directed bispecific antibodies in B-cell lymphoma. Blood, 96: 3544-3552, 2000.

21. Deo, Y. M., Graziano, R F., Repp, R., and van de Winkel, J. G. Clinical significance of IgG Fc receptors and FcyR-directed immunotherapies. Immunol.Today, 18: 127-135, 1997.

22. Ravetch, J. V. and Kinet. J. P. Fc receptors. Annu.Rev.Immunol., 9: 457-492, 1991.

23. van de Winkel, J. G. and Capel, P. J. Human IgG Fc receptor heterogeneity: molecular aspects and clinical implications. Immunol. Today. 14: 215-221, 1993.

24. Valerius, T., Repp, R, de Wit, T. P., Berthold, S., Platzer, E., Kalden, J. R, Gramatzki, M., and van de Winkel, J. O. Involvement of the high-affinity receptor for IgG (FcyRI; CD64) in enhanced tumor cell cytotoxicity of neutrophils during granulocyte colony-stimulating factor therapy. Blood, 82: 931-939, 1993.

25. Cella, M., F. Sallusto, and A. Lanzavecchia. 1997. Origin, maturation and antigen-presenting function of dendritic cells. Curr. Opin. Immunol. 9:10.

26. Rock, K. L. 1996. A new foreign policy: MHC class I molecules monitor the outside world. Immunol. Today 17:131.

27. Brossart, P. and M. J. Bevan. 1997. Presentation of exogenous protein antigens on major histocompatibility complex class I molecules by dendritic cells: pathway of presentation and regulation by cytokines. Blood 90:1594.

28. Rock, K. L., S. Gamble, and L. Rothstein. 1990. Presentation of exogenous antigen with class I major histocompatibility complex molecules. Science 249:918.

29. Mitchell, D. A., S. K. Nair, and E. Gilboa. 1998. Dendritic cell/macrophage precursors capture exogenous antigen for MHC class I presentation by dendritic cells. Eur. J. Immunol. 28:1923.

30. Regnault, A., D. Lankar, V. Lacabanne, A. Rodriguez, C. Thery, M. Rescigno, T. Saito, S. Verbeek, C. Bonnerot, P. Ricciardi-Castagnoli, and S. Amigorena. 1999. Fcγ receptor-mediated induction of dendritic cell maturation and major histocompatibility complex class I-restricted antigen presentation after immune complex internalization. J Exp. Med. 189:371.

31. van Vugt, M. J., and J. G. J. van de Winkel. 2000. Fc receptors. In Embryonic Encyclopedia of Life Sciences. Nature Publishing Group,. www.els.net, London.

32. Heijnen, I. A. F. M., M. J. van Vugt, N. A. Fanger, R. F. Graziano, T. P. de Wit, F. M. Hofhuis, P. M. Guyre, P. J. A. Capel, J. S. Verbeek, and J. G. J. van de Winkel. 1996. Antigen targeting to myeloid-specific human Fc□RI/CD64 triggers enhanced antibody responses in transgenic mice. J. Clin. Invest 97:331.

33. Guyre, P. M., R. F. Graziano, J. Goldstein, P. K. Wallace, P. M. Morganelli, K. Wardwell, and A. L. Howell. 1997. Increased potency of Fc-receptor-targeted antigens. Cancer Immunol. Immunother. 45:146.

34. van Vugt, M. J., M. J. Kleijmeer, T. Keler, I. Zeelenberg, M. A. van Dijk, J. H. W. Leusen, H. J. Geuze, and J. G. J. van de Winkel. 1999. The FcγRIa (CD64) ligand binding chain triggers major histocompatibility complex class II antigen presentation independently of its associated FcR γ-chain. Blood 94:808.

35. Keler, T., P. M. Guyre, L. A. Vitale, K. Sundarapandiyan, J. G. J. van de Winkel, Y. M. Deo, and R. F. Graziano. 2000. Targeting weak antigens to CD64 elicits potent humoral responses in human CD64 transgenic mice. J. Immunol. 165:6738.

36. Guyre, C. A., M. E. Barreda, S. L. Swink, and M. W. Fanger. 2001. Colocalization of FcγRI-targeted antigen with class I MHC: implications for antigen processing. J. Immunol. 166:2469.

37. Bird, A. P. 1987. CpG islands as gene markers in the vertebrate nucleus. Trends genet. 3:342.

38. Krieg, A. M., A. K. Yi, S. Matson, T. J. Waldschmidt, G. A. Bishop, R. Teasdale, G. A. Koretzky, and D. M. Klinman. 1995. CpG motifs in bacterial DNA trigger direct B-cell activation. Nature 374:546.

39. Liu, H. M., S. E. Newbrough, S. K. Bhatia, C. E. Dahle, A. M. Krieg, and G. J. Weiner. 1998. Immunostimulatory CpG oligodeoxynucleotides enhance the immune response to vaccine strategies involving granulocyte-macrophage colony-stimulating factor. Blood 92:3730.

40. Sparwasser, T., E. S. Koch, R. M. Vabulas, K. Heeg, G. B. Lipford, J. W. Ellwart, and H. Wagner. 1998. Bacterial DNA and immunostimulatory CpG oligonucleotides trigger maturation and activation of murine dendritic cells. Eur. J. Immunol. 28:2045.

41. Hartmann, G., G. J. Weiner, and A. M. Krieg. 1999. CpG DNA: a potent signal for growth, activation, and maturation of human dendritic cells. Proc. Natl. Acad. Sci. U.S.A 96:9305.

42. Vabulas, R. M., H. Pircher, G. B. Lipford, H. Hacker, and H. Wagner. 2000. CpG-DNA activates in vivo T cell epitope presenting dendritic cells to trigger protective antiviral cytotoxic T cell responses. J. Immunol. 164:2372.

43. Graziano, R. F., P. R. Tempest, P. White, T. Keler, Y. M. Deo, H. Ghebremariam, K. Coleman, L. C. Pfefferkorn, M. W. Fanger, and P. M. Guyre. 1995. Construction and characterization of a humanized anti-γ-Ig receptor type I (FcγRI) monoclonal antibody. J. Immunol. 155:4996.

44. Grant, E. P. and K. L. Rock. 1992. MHC class I-restricted presentation of exogenous antigen by thymic antigen-presenting cells in vitro and in vivo. J. Immunol. 148:13.

45. Shimonkevitz, R., J. Kappler, P. Marrack, and H. Grey. 1983. Antigen recognition by H-2-restricted T cells. I. Cell-free antigen processing. J. Exp. Med. 158:303.

46. Gillis, S., M. M. Ferm, W. Ou, and K. A. Smith. 1978. T cell growth factor: parameters of production and a quantitative microassay for activity. J. Immunol. 120:2027.

47. Inaba, K., W. J. Swiggard, M. Inaba, J. Meltzer, A. Mirza, T. Sasagawa, M. C. Nussenzweig, and R. M. Steinman. 1995. Tissue distribution of the DEC-205 protein that is detected by the monoclonal antibody NLDC-145. I. Expression on dendritic cells and other subsets of mouse leukocytes. Cell Immunol. 163:148.

48. Bhattacharya, A., M. E. Dorf, and T. A. Springer. 1981. A shared alloantigenic determinant on Ia antigens encoded by the I-A and I-E subregions: evidence for I region gene duplication. J. Immunol. 127:2488.

49. Graziano, R. F., C. Somasunduram, and J. Goldstein. 1998. The production of bispecific antibodies. In Bispecific Antibodies. Springer-Verlag, New York, pp. 1.

50. Inaba, K., M. Inaba, N. Romani, H. Aya, M. Deguchi, S. Ikehara, S. Muramatsu, and R. M. Steinman. 1992. Generation of large numbers of dendritic cells from mouse bone marrow cultures supplemented with granulocyte/macrophage colony-stimulating factor. J. Exp. Med. 176:1693.

51. Chen, B., Y. Shi, J. D. Smith, D. Choi, J. D. Geiger, and J. J. Mule. 1998. The role of tumor necrosis factor □ in modulating the quantity of peripheral blood-derived, cytokine-driven human dendritic cells and its role in enhancing the quality of dendritic cell function in presenting soluble antigens to CD4+T cells in vitro. Blood 91:4652.

52. Labeur, M. S., B. Roters, B. Pers, A. Mehling, T. A. Luger, T. Schwarz, and S. Grabbe. 1999. Generation of tumor immunity by bone marrow-derived dendritic cells correlates with dendritic cell maturation stage. J. Immunol. 162:168.

53. te Velde, A. A., M. R. de Waal, R. J. Huijbens, J. E. de Vries, and C. G. Figdor. 1992. IL-10 stimulates monocyte Fc□ R surface expression and cytotoxic activity. Distinct regulation of antibody-dependent cellular cytotoxicity by IFN-gamma, IL-4, and IL-10. J. Immunol. 149:4048.

54. Fanger, N. A., D. Voigtlaender, C. Liu, S. Swink, K. Wardwell, J. Fisher, R. F. Graziano, L. C. Pfefferkom, and P. M. Guyre. 1997. Characterization of expression, cytokine regulation, and effector function of the high affinity IgG receptor Fc RI (CD64) expressed on human blood dendritic cells. J. Immunol. 158:3090.

55. Sallusto, F. and A. Lanzavecchia. 1994. Efficient presentation of soluble antigen by cultured human dendritic cells is maintained by granulocyte/macrophage colony-stimulating factor plus interleukin 4 and downregulated by tumor necrosis factor alpha. J. Exp. Med. 179:1109.

56. Warren, T. L., S. K. Bhatia, A. M. Acosta, C. E. Dahle, T. L. Ratliff, A. M. Krieg, and G. J. Weiner. 2000. APC stimulated by CpG oligodeoxynucleotide enhance activation of MHC class I-restricted T cells. J. Immunol. 165:6244.

57. Liu, C., J. Goldstein, R. F. Graziano, J. He, J. K. O'Shea, Y. M. Deo, and P. M. Guyre. 1996. F(c)γRI-targeted fusion proteins result in efficient presentation by human monocytes of antigenic and antagonist T cell epitopes. J. Clin. Invest 98:2001.

58. van de Winkel, J. G. J. and P. J. A. Capel. 1993. Human IgG Fc receptor heterogeneity: molecular aspects and clinical implications. Immunol. Today 14:215.

59. Wallace, P. K., K. Y. Tsang, J. Goldstein, P. Correale, T. M. Jarry, J. Schlom, P. M. Guyre, M. S. Ermstoff, and M. W. Fanger. 2001. Exogenous antigen targeted to Fc□RI on myeloid cells is presented in association with MHC class I. J. Immunol. Methods 248:183.

60. Honeychurch, J., A. L. Tutt, T. Valerius, I. A. F. M. Heijnen, J. G. J. van de Winkel, and M. J. Glennie. 2000. Therapeutic efficacy of Fc□RI/CD64-directed bispecific antibodies in B-cell lymphoma. Blood 96:3544.

61. van Spriel, A. B., I. E. van den Herik-Oudijk, and J. G. J. van de Winkel. 2001. Cutting Edge: Neutrophil FcγRI as target for immunotherapy of invasive candidiasis. J. Immunol. 166:7019.

62. Harrison, P. T., W. Davis, J. C. Norman, A. R. Hockaday, and J. M. Allen. 1994. Binding of monomeric immunoglobulin G triggers FcγRI-mediated endocytosis. J. Biol. Chem. 269:24396.

63. Machy, P., K. Serre, and L. Leserman. 2000. Class I-restricted presentation of exogenous antigen acquired by Fcγreceptor-mediated endocytosis is regulated in dendritic cells. Eur J. Immunol. 30:848.

64. Banchereau, J., and R. M. Steinman. 1998. Dendritic cells and the control of immunity. Nature 392:245.

65. Bachmann, M. F., M. B. Lutz, G. T. Layton, S. J. Harris, T. Fehr, M. Rescigno, and P. Ricciardi-Castagnoli. 1996. Dendritic cells process exogenous viral proteins and virus-like particles for class I presentation to CD8+cytotoxic T lymphocytes. Eur. J. Immunol. 26:2595.

Incorporation by Reference

All patents, pending patent applications and other publications cited herein are hereby incorporated by reference in their entirety.

Equivalents

Those skilled in the art will recognize, or be able to ascertain using no more than routine experimentation, many equivalents to the specific embodiments of the invention described herein. Such equivalents are intended to be encompassed by the following claims.

We claim:

1. A composition, comprising one or more CpG-containing oligodeoxynucleotides in combination with a multispecific molecule which binds to an Fc receptor and a target antigen.

2. The composition of claim 1, wherein the multispecific molecule binds to a human Fcγ receptor.

3. The composition of claim 2, wherein the Fcγ receptor is selected from the group consisting of FcγRI (CD64), FcγRII (CD32), and FcγRIII (CD16).

4. The composition of claim 2, wherein the Fcγ receptor is FcγRI (CD64).

5. The composition of claim 1, wherein the multispecific molecule comprises a bispecific antibody.

6. The composition of claim 1, wherein the target antigen is a tumor cell.

7. The composition of claim 6, wherein the tumor cell is selected from the group consisting of ovarian, breast, testicular, prostate, leukemia, and lymphoma tumor cells.

8. The composition of claim 1, wherein the target antigen is a pathogen.

9. The composition of claim 8, wherein the pathogen is a virus or a bacterium.

10. The composition of claim 1, wherein the composition enhances Fc receptor-mediated antibody dependent cellular cytotoxicity (ADCC) of a cell expressing the target antigen in the presence of an effector cell.

11. The composition of claim 10, wherein the effector cell is selected from the group consisting of a neutrophil, a monocyte, a macrophage, and a polymorphonuclear (PMN) cell.

12. The composition of claim 11, wherein the effector cell is a neutrophil.

13. The composition of claim 12, wherein expression of FcγRI (CD64) is upregulated on the neutrophil.

14. The composition of claim 10, wherein the cell is a lymphoma cell.

15. The composition of claim 14 further comprising a chemotherapeutic agent.

16. The composition of claim 1, wherein the composition enhances Fc receptor-mediated antigen presentation of a cell expressing the target antigen.

17. The composition of claim 1, wherein the composition enhances dendritic cell-mediated cross-presentation of an Fc receptor-targeted antigen.

18. The composition of claim 1, wherein the multispecific molecule comprises an antibody which binds to an Fc receptor at a site which is distinct from the natural ligand binding site of the receptor.

19. The composition of claim 1, wherein the multispecific molecule comprises an antibody fragment or a single chain antibody.

20. The composition of claim 1, wherein the multispecific molecule comprises a human antibody or fragment thereof.

21. A vaccine composition, comprising one or more CpG-containing oligodeoxynucleotides in combination with an FcR-targeted antigen.

22. The composition of claim 21, wherein the antigen is selected from the group consisting of a tumor antigen, a viral antigen and a bacterial antigen.

23. The composition of claim 22, wherein the tumor antigen is selected from the group consisting of ovarian, breast, testicular, prostate, leukemia, and lymphoma tumor antigens.

24. The composition of claim 21, wherein the Fc receptor is a human Fcγ receptor.

25. The composition of claim 24, wherein the Fcγ receptor is selected from the group consisting of FcγRI (CD64), FcγRII (CD32), and FcγRIII (CD16).

26. The composition of claim 24, wherein the Fcγ receptor is FcγRI (CD64).

27. The composition of claim 21, wherein the FcR-targeted antigen comprises a fusion protein.

28. The composition of claim 21 further comprising a chemotherapeutic agent.

29. The composition of claim 21, wherein the composition enhances Fc receptor-mediated antigen presentation of a cell expressing the target antigen.

30. The composition of claim 28, wherein the effector cell is selected from the group consisting of a neutrophil, a monocyte, a macrophage, and a polymorphonuclear (PMN) cell.

31. The composition of claim 30, wherein the effector cell is a neutrophil.

32. The composition of claim 32, wherein expression of FcγRI (CD64) is upregulated on the neutrophil.

33. The composition of claim 29, wherein the cell is a lymphoma cell.

34. The composition of claim 21, wherein the composition enhances dendritic cell-mediated cross-presentation of the Fc receptor-targeted antigen.

35. The composition of claim 21, wherein the FcR-targeted antigen comprises an antibody which binds to an Fc receptor at a site which is distinct from the natural ligand binding site of the receptor.

36. The composition of claim 21, wherein the FcR-targeted antigen comprises an antibody fragment or a single chain antibody.

37. The composition of claim 21, wherein the FcR-targeted antigen comprises a human antibody or fragment thereof.

38. A method of enhancing Fc receptor-mediated ADCC of a target cell comprising administering to a subject a composition comprising one or more CpG-containing oligodeoxynucleotides in combination with a multispecific molecule which binds to an Fc receptor and a target antigen.

39. A method of inhibiting the growth of a target cell comprising administering to a subject a composition comprising one or more CpG-containing oligodeoxynucleotides in combination with a multispecific molecule which binds to an Fc receptor and a target antigen.

40. The method of claim 38, further comprising the administration of a chemotherapeutic agent.

41. The method of claim 40, wherein the chemotherapeutic agent is selected from the group consisting of doxorubicin (adriamycin), cisplatin bleomycin sulfate, carmustine, chlorambucil, and cyclophosphamide hydroxyurea.

42. The method of claim 38, further comprising the administration of radiation therapy.

43. The method of claim 38, further comprising the administration of a cytokine.

44. The method of claim 43, wherein the cytokine is selected from the group consisting of granulocyte colony-stimulating factor (G-CSF), granulocyte-macrophage colony-stimulating factor (GM-CSF), interferon-γ (IFN-γ), and tumor necrosis factor (TNF).

45. The method of claim 38, wherein the Fcγ receptor is FcγRI (CD64).

46. The method of claim 38, wherein the multispecific molecule comprises a bispecific antibody.

47. The method of claim 38, wherein the target antigen is a tumor cell.

48. The method of claim 38, wherein the a tumor cell is a lymphoma cell.

49. The method of claim 38, wherein the target antigen is a pathogen.

50. The method of claim 38, wherein the multispecific molecule comprises a human antibody or fragment thereof.

51. A method for enhancing Fc receptor-mediated antigen presentation, comprising administering a vaccine composition comprising one or more CpG-containing oligodeoxynucleotides in combination with an FcR-targeted antigen.

52. The method of claim 51, wherein the antigen is selected from the group consisting of a tumor antigen, a viral antigen and a bacterial antigen.

53. The method of claim 51, wherein the Fcγ receptor is FcγRI (CD64).

54. The method of claim 51, wherein the FcR-targeted antigen comprises a fusion protein.

55. The method of claim 51, wherein the composition enhances Fc receptor-mediated antigen presentation of a cell expressing the target antigen.

56. The method of claim 51, further comprising the administration of a chemotherapeutic agent.

57. The method of claim 51, further comprising the administration of radiation therapy.

58. The method of claim 51, further comprising the administration of a cytokine.
